(19) United States
(12) Patent Application Publication
(10) Pub. No.: US 2009/0011013 A1
(43) Pub. Date: Jan. 8, 2009

(54) Tacrolimus Combination Products

(75) Inventors: Lisbet Bonlokke Ranklove, Holte (DK); Jan Moller Mikkelsen, Gentofte (DK)

Correspondence Address: DARBY & DARBY P.C., P.O. BOX 770, Church Street Station, New York, NY, 10008-0770, US

(73) Assignee: LIFECYCLE PHARMA A/S, Horsholm (DK)

(21) Appl. No.: 12/089,082

(22) PCT Filed: Oct. 9, 2006
(86) PCT No.: PCT/DK2006/000569

§ 371 Date: May 19, 2008

(30) Foreign Application Priority Data
Oct. 7, 2005 (DK) PA 2005 01411

(51) Int. Cl. A61K 9/20; A61K 31/439; A61K 31/496; A61P 37/00; A61K 31/7048; A61K 9/14

(52) U.S. Cl. 424/464; 514/291; 514/254.07; 514/29; 424/490

(57) ABSTRACT

The present invention relates to a pharmaceutical composition comprising tacrolimus or an analogue thereof and a substance being a substrate for CYP3A4 and/or P-glycoprotein, oral solid dosage forms comprising the pharmaceutical composition such as tablets, methods for preparing the pharmaceutical composition and oral dosage forms and use of the pharmaceutical composition for preparing a medicament. The substance being a substrate for CYP3A4 and/or P-glycoprotein is preferably cyclosporine A. The invention further relates to treatment of a patient in need thereof by coadministration of the combination according to the invention. In a further aspect, the invention relates to the above combination further comprising a CYP3A4 inhibitor compound, preferably a compound naturally occurring in citrus juice, for example grapefruit juice, preferably a spiro ortho ester compound.

The present invention relates to a pharmaceutical composition comprising tacrolimus or an analogue thereof and a substance being a substrate for CYP3A4 and/or P-glycoprotein, oral solid dosage forms comprising the pharmaceutical composition such as tablets, methods for preparing the pharmaceutical composition and oral dosage forms and use of the pharmaceutical composition for preparing a medicament. The substance being a substrate for CYP3A4 and/or P-glycoprotein is preferably cyclosporine A. The invention further relates to treatment of a patient in need thereof by coadministration of the combination according to the invention. In a further aspect, the invention relates to the above combination further comprising a CYP3A4 inhibitor compound, preferably a compound naturally occurring in citrus juice, for example grapefruit juice, preferably a spiro ortho ester compound.

### BACKGROUND OF THE INVENTION

Tacrolimus, also known as FK-506 or FR-900506, has the chemical tricyclic structure shown below:

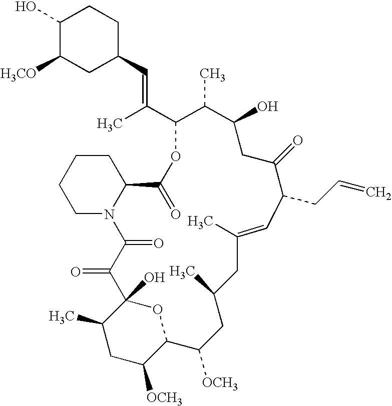

corresponding to C₄₄H₆₉NO₁₂. Tacrolimus appears in the form of white crystals or crystalline powder. It is practically insoluble in water, freely soluble in ethanol and very soluble in methanol and chloroform.

The preparation of tacrolimus is described in EP-A-0 184 162 and analogues of tacrolimus are disclosed e.g. in EP-A-0 444 659 and U.S. Pat. No. 6,387,918, which are both hereby incorporated by reference. Improved pharmaceutical formulations of tacrolimus with improved bioavailability, for example solid solutions of tacrolimus, are disclosed in WO2005/020993 and WO2005/020994 both of which are hereby incorporated by reference.

Tacrolimus is a macrolide compound with useful immunosuppressive activity, antimicrobial activity and other pharmacological activities and is of value for the treatment or prevention of rejection reactions by transplantation of organs or tissues, graft versus host diseases, autoimmune diseases and infectious diseases. Tacrolimus prolongs the survival of the host and transplanted graft in animal transplant models of liver, kidney, heart, bone marrow and small bowel and pancreas, lung and trachea, skin, cornea and limb.

In animals, tacrolimus has been demonstrated to suppress some humoral immunity and, to a greater extent, cell-mediated reactions such as allograft rejection, delayed type hypersensitivity, collagen-induced arthritis, experimental allergic encephalomyelitis and graft-versus-host disease.

Tacrolimus inhibits T-lymphocyte activation, although the exact mechanism of action is unknown. Experimental evidence suggest that tacrolimus binds to an intracellular protein, FKBP-12. A complex of tacrolimus-FKBP-12, calcium, calmodulin, and calcineurin is then formed and the phosphatase activity of calcineurin inhibited. This effect may prevent the dephosphorylation and translocation of nuclear factor of activated T-cells, a nuclear component thought to initiate gene transcription for the formation of lymphokines. The net result is the inhibition of T-lymphocyte activation, i.e. immunosuppression.

Usually tacrolimus is administered orally and is therefore absorbed from the gastrointestinal tract. It has been observed that the absorption is negatively influenced by the simultaneous ingestion of food. Thus, the rate and extent of tacrolimus absorption were greatest under fasted conditions.

In general, it is known that the absorption and bioavailability of a therapeutically active substance can be affected by a variety of factors when administered orally. Such factors include the presence of food in the gastrointestinal tract and, in general, the gastric residence time of a drug substance is significantly longer in the presence of food than in the fasted state. If the bioavailability of a drug substance is affected beyond a certain point due to the presence of food in the gastrointestinal tract, the drug substance is said to exhibit a food effect. Food effects are important because absorption and hence the plasma levels becomes highly variable depending on food intake. Absorption into the bloodstream may be adversely affected to the point that the patient risks insufficient absorption to remedy the condition for which the drug was administered. On the other hand, the very high peak concentrations occasionally observed at fasted conditions may very well induce significant side effects of nephro- or neuro-toxic origin as well as GI side-effects and others.

Absorption of tacrolimus from the gastrointestinal tract after oral administration is rapid with a mean time-to-peak concentration (tmax) of approximately 1-2 hours after administration to healthy subjects or kidney or liver transplanted patients, but incomplete and variable. The bioavailability is generally as low as at the most about 20% after oral administration. This phenomenon is sometimes also denoted the “first-pass effect”, i.e. the process of drug degradation during a drug's transition from initial ingestion to circulation in the blood stream.

Frequently observed side effects are vomiting and nausea but side effects like tremor, headache, hypertension, renal dysfunction, hyperkalemia, hypomagnesaemia, hyperglycemia, insomnia, diarrhea, constipation, abdominal pain, nephrotoxicity and neurotoxicity are also observed.

For oral administration, tacrolimus is currently formulated and marketed as soft gelatine capsules comprising the equivalent of 0.5, 1 or 5 mg anhydrous tacrolimus and marketed under the trade name Prograf® and Protropic®. The recommended initial oral dose is from about 0.1 to 0.2 mg/kg/day in patients. The dose aims at a certain trough plasma level from about 5 to about 20 ng/ml. Prograf® is indicated for the prophylaxis of organ rejection in patients receiving allogeneic liver or kidney transplants.

Tacrolimus is extensively metabolized by the CYP3A4 isoenzyme in the gut wall and liver. Therefore, drugs that affect this isoenzyme may influence absorption and the subsequent elimination of systemically absorbed tacrolimus. Inhibitors of CYP3A4 may increase tacrolimus levels, while inducers of CYP3A4 may increase the metabolism of tacrolimus and decrease tacrolimus levels. It has been suggested to administer tacrolimus together with one or more CYP3A4 inhibitors in order to improve the overall bioavailability.

In order to increase the bioavailability of tacrolimus, prior art has focused on agents capable of inhibiting the cytochrome P450 system. Inhibition of the P450 system is a model for in vitro determination of in vivo bioavailability enhancement. See, e.g., U.S. Pat. Nos. 5,478,723 and 5,567,592, both incorporated herein by reference, for a more full description of the P450 system. It has been found that the following compounds may be inhibitors of the cytochrome P450 system, i.e. are CYP3A4 inhibitors: ketoconazole, fluconazole, ritonavir, itraconazole, miconazole, erythromycin and troleandomycin. In addition hereto, it is well established that the intake of grapefruit juice may also enhance the bioavailability of certain drugs. Based on this known effect, a number of compounds present in citrus juice including grapefruit juice have been identified and synthesized as disclosed in the international publications WO98/53658, WO99/09976, WO00/54768, WO0/00042 and WO2004/037827 which are all incorporated herein by reference. Especially, these publications disclose a number of spiro ortho esters and spiro ortho carbonates as CYP3A4 inhibitors.

Cyclosporine, the active principle in Sandimmune® (cyclosporine) and Neoral® both commercially available in US and Sandimmune Neoral®I commercially available I Europe and corresponding to the product Neoral® is a cyclic polypeptide immunosuppressant agent consisting of 11 amino acids. It is produced as a metabolite by the fungus species Beauveria nlyea.

Chemically, cyclosporine is designated as [R-[R*,R*-(E)]]-cyclic(L-alanyl-D-alanyl-N-methyl-L-leucyl-N-methyl-L-leucyl-N-methyl-L-valyl-3-hydroxy-N, 4-dimethyl-L-2-amino-6-octenoyl-L-α-amino-butyryl-N-methylglycyl-N-methyl-L-leucyl-L-valyl-N-methyl-L-leucyl).

Cyclosporine is a potent immunosuppressive agent which in animals prolongs survival of allogeneic transplants involving skin, heart, kidney, pancreas, bone marrow, small intestine, and lung. Cyclosporine has been demonstrated to suppress some humoral immunity and to a greater extent, cell-mediated reactions such as allograft rejection, delayed hypersensitivity, experimental allergic encephalomyelitis, Freunds adjuvant arthritis, and graft vs. host disease in many animal species for a variety of organs.

Successful kidney, liver, and heart allogeneic transplants have been performed in man using Sandimmune® (cyclosporine). The exact mechanism of action is not known. Experimental evidence suggests that the effectiveness of cyclosporine is due to specific and reversible inhibition of immunocompetent lymphocytes in the G0- or G1-phase of the cell cycle. T-lymphocytes are preferentially inhibited. The 1-helper cell is the main target, although the 1-suppressor cell may also be suppressed. Cyclosporine also inhibits lymphokine production and release including interleukin-2 or 1-cell growth factor (TCGF). In the present context cyclosporin A, cyclosporine A, and CsA will be used synonymously. Sandimmune Neoral® and Neoral® is actually identical products while Sandimmune® an older product have a slower absorption rate and less bioavailability.

The absorption of cyclosporine from the gastrointestinal tract is incomplete and variable. Peak concentration (Cmax) in blood and plasma for the Sandimmune® product is achieved at about 3.5 hours and within 1-2 hours for the Sandimmune Neoral® and Neoral® products. Cmax and area under the plasma or blood concentration/time curve (AUC) increase with the administered dose; for blood the relationship is curvilinear (parabolic) between 0 and 1400 mg.

As determined by a specific assay, Cₘₐₓ of Sandimmune® is approximately 1.0 ng/mL/mg of dose for plasma and 2.7-1.4 ng/mL/mg of dose for blood (for low to high doses). Compared to an intravenous infusion, the absolute bioavailability of the oral solution is approximately 30% based upon the results in 2 patients. The bioavailability of Sandimmune® soft gelatin capsules (cyclosporine capsules, USP) is equivalent to Sandimmune® oral solution, (cyclosporine oral solution, USP).

Cyclosporine is distributed largely outside the blood volume. In blood the distribution is concentration dependent. Approximately 33%-47% is in plasma, 4%-9% in lymphocytes, 5%-12% in granulocytes, and 41%-58% in erythrocytes. At high concentrations, the uptake by leukocytes and erythrocytes becomes saturated. In plasma, approximately 90% is bound to proteins, primarily lipoproteins.

The disposition of cyclosporine from blood is biphasic with a terminal half-life of approximately 19 hours (range: 10-27 hours). Elimination is primarily biliary with only 6% of the dose excreted in the urine.

Cyclosporine is extensively metabolized but there is no major metabolic pathway. Only 0.1% of the dose is excreted in the urine as unchanged drug. Of 15 metabolites characterized in human urine, 9 have been assigned structures. The major pathways consist of hydroxylation of the C-g-carbon of 2 of the leucine residues, C1-carbon hydroxylation, and cyclic ether formation (with oxidation of the double bond) in the side chain of the amino acid 3-hydroxyl-N,4-dimethyl-L-2-amino-6-octenoic acid and N-demethylation of N-methyl leucine residues. Hydrolysis of the cyclic peptide chain or conjugation of the aforementioned metabolites do not appear to be important biotransformation pathways

The absorption and metabolic pattern for tacrolimus has been extensive investigated and “Effects of Intestinal and Hepatic Metabolism on the Bioavailability of Tacrolimus in Rats” (Yukiya Hashimonto et al. Pharmaceutical Research, Vol. 15, No. 10, 1998) demonstrates rapid rate of absorption of tacrolimus in the intestine and almost complete absorption. After intraportal (infusion over a 1-hour period) administration the bioavailability was about 40% and 25% after intestinal administration and it is concluded that the metabolism of tacrolimus in the intestines contributes to its extensive and variable first pass metabolism following the oral administration. The hepatic extraction ratio was calculated to 60% and intestinal extraction to 34%. It is mentioned that the first pass effect in the intestines may be more related to metabolism by CYP3A than to drug exsorption by P-glycoprotein and that the P-glycoprotein may cause a shift in drug location to the distal small intestine which contain a lower amounts of CYP3A4, and that P-glycoprotein may prolong the duration of absorption. Thus, P-glycoprotein may affect the extraction of drugs in the intestine. In “Roles of the Jejunum and Ileum in the First-Pass Effect as absorptive Barriers for Orally Administered Tacolimus. Masahiro Shimonura et al. (Journal of Surgical Research 103, 215-222 (2002)) it is mentioned that the first-pass effect of tacrolimus in the small intestines shows regional differences with highest metabolism in the duodenum followed by jejunum and ileum, respectively. This is counter acted by the higher expression of P-glycoprotein in the ileum. In addition, it is demonstrated that the time to Cₘₐₓ is twofold higher when jejunum is resected (absorption in the ileum) compared to ileum-resected rats (absorption in jejunum). In other words, the presence of the P-glycoprotein efflux may decrease the apparent absorption rate. In “Characterization of Interintestinal and Intraintestinal Variation in Human CYP3A-Dependent Metabolism, Mary F. Paine et al (The Journal of Pharmacology and Experimental Therapeutics, JPET 283:1552-1562, 1997) the presence of CYP3A and activity in the human intestine is demonstrated toward the probe midazolam. Estimated distribution in duodenum/jejunum/ileum was 44%/32%/24% of the median content in total liver CYP3A. The content of CYP3A protein is not always correlated to activity of the enzyme. It is concluded that the intervariability in absorption observed for CYP3A drugs is due to the high intervariation in CYP3A. In “Combined Therapy with FK-506 and Cyclosporine for Canine Lung Allotransplantation Immunosuppressive Effects and Blood Trough Levels

Tatsuo Fukuse et al” (The Journal of Heart and Lung Transplantation: Vol 12, No 6, Part 1; November/December 1993: pp 941-947) use of tacrolimus and CsA alone or together in dogs are tested in 6 different groups and receiving oral or intramuscular dosages. One group received standard doses resulting in adequate immunosuppression of: A. Tacrolimus optimal dose i.m. 0.1 mg/kg/day and B. CsA optimal dose orally 20 mg/kg/day. A second group received a suboptimal dose resulting in rejection of: C. Tacrolimus i.m. 0.03 mg/kg/day and D. CsA orally 6 mg/kg/day. The third group received a third (E) and a half (F) of individual standard dose in combination of: E. Tacro 0.03 mg/kg/day+CsA 6 mg/kg/day resulting in insufficient, however additional effect compared with individual doses from C and D, respectively and F. Tacro 0.05 mg/kg/day+CsA 10 mg/kg/day resulting in effect equal to or superior to A and B, respectively. The blood concentration of tacrolimus in group E was not significant higher than when given alone in same dose in group C. Similarly, no significant difference in CsA concentration was seen between single and combination treatment of D and E, respectively. In “Disposition of Tacrolimus in Isolated Perfused Rat Liver: Influence of Troleandomycin, Cyclosporine, and GG918, ChiYuan Wu et al (Drug Metabolism and Disposition 31:1292-1295, 2003) it is mentioned that the considerable overlap in substrate selectivity makes it difficult to differentiate the interactive roles of P-gp and CYP3A in xenobiotic disposition. In “Pharmacokinetics of FK 506 Following Oral Administration: A Comparison of FK 506 and Cyclosporine. R Venkatarmanan et all. (Transplantation Proceedings, Vol 23 No 1 (February) 1991: pp 931-933) it is demonstrated that most patients reach peak within 1 hour, however some not before 4 hours. In half of 14 patients there was sustained plasma concentration throughout the entire dosing interval, the suggested cause is slow dissolution of tacrolimus from the solid dispersion in the gastric fluids. Bioavailability ranged from 6% to 56%.

It appears that treatment with the immunosuppressant substances tacrolimus results in low and often unpredictable bioavailability. This results in a risk of either low efficacy or high toxicity. Accordingly, there is an unmet need for novel pharmaceutical compositions and/or dosage forms comprising tacrolimus exhibiting enhanced bioavailability and higher predictability. An increased bioavailability may allow a reduction in the dosage units taken by a patient, e.g. down to a single dose daily, and may also reduce the need to be administered in a specific and constant time relation to food intake thereby allowing patients more freedom on when and how the drug is taken. Furthermore, it is contemplated that fluctuations in the plasma concentration versus time profile may be significantly reduced avoiding periods with insufficient exposure and periods with toxic exposure. Further, enhanced bioavailability may also result in a more reproducible (i.e. less variable compared to that of Prograf®) release profile.

### SUMMARY OF THE INVENTION

The inventors have surprisingly found that co-administration of tacrolimus and a substance being a substrate for CYP3A4 and/or P-glycoprotein such as cyslosporin A and optionally an addition of a further CYP3A4 inhibitor can lead to in an improvement of the bioavailability of tacrolimus when compared with administration of tacrolimus without a substance being a substrate for CYP3A4 and/or P-glycoprotein.

Accordingly, in a first aspect the present invention relates to treatment of a patient in need thereof with an oral pharmaceutical dosage form comprising tacrolimus or an analogue thereof in combination with a substance being a substrate for CYP3A4 and/or P-glycoprotein such as cyclosporine A, wherein the treatment upon administration to the human in need thereof results in a relative AUCᵢₙᵥₑₙₜᵢₒₙ/AUCₛₑₚₐᵣₐₜₑ value of tacrolimus of at least about 1.1, the AUC values being determined under similar conditions both with respect to the administration regime and with respect to the oral dosage form.

It is to be understood, that the invention also encompasses a pharmaceutical composition comprising the combination, wherein the composition upon administration to a human in need thereof results in a relative AUCᵢₙᵥₑₙₜᵢₒₙ/AUC_{per se} value of tacrolimus of at least about 1.1, the AUC values being determined under similar conditions and the AUC_{per se} value is determined after administration of tacrolimus per se (without the substance being a substrate for CYP3A4 and/or P-glycoprotein).

In a second aspect, the invention relates to a dosage form, typically a solid unit dosage form for oral administration, comprising the pharmaceutical composition of the invention.

In a further aspect, the invention relates to a single solid dosage form for oral administration comprising a first solid pharmaceutical composition containing tacrolimus or an analogue thereof as the active substance and second solid pharmaceutical composition containing a substance being a substrate for CYP3A4 and/or P-glycoprotein as the active substance, wherein the first and the second pharmaceutical composition are present in separate entities. This single solid dosage form may be prepared by a method comprising the steps of: i) preparing the first solid pharmaceutical composition, ii) preparing the second solid pharmaceutical composition, and iii) compressing the first and second compositions into a multilayer tablet, the first and second compositions being present in separate layers.

In a further aspect, the invention relates to a single solid dosage form suitable for oral administration comprising tacrolimus or an analogue thereof as the active substance and second solid pharmaceutical composition containing a substance being a substrate for CYP3A4 and/or P-glycoprotein as the active substance, wherein the tacrolimus is present as component of an immediate release pharmaceutical formulation and the substance being a substrate for CYP3A4 and/or P-glycoprotein is present as component of either an immediate release pharmaceutical formulation or a delayed release pharmaceutical formulation. Accordingly, in one aspect both substances is in immediate form, or both in a delayed or controlled form, or combinations thereof.

In yet another aspect, the invention relates to a method for the preparation of the pharmaceutical composition, the method comprising the steps of dissolving or dispersing tacrolimus in a solid, hydrophilic or water-miscible vehicle to obtain a solid dispersion or a solid solution or a mixture thereof, followed by mixing the substance being a substrate for CYP3A4 and/or P-glycoprotein with the solid dispersion or solid solution.

In yet another aspect, the invention relates to use of the pharmaceutical composition of the invention for preparing a medicament. In a still further aspect, the invention relates to a method for treating a patient in need thereof by coadministration of tacrolimus with a substance being a substrate for CYP3A4 and/or P-glycoprotein.

### DETAILED DESCRIPTION OF THE INVENTION

### Definitions

As used herein, the term “active substance”, “active pharmaceutical substance”, “active ingredient” or “active pharmaceutical ingredient” means any component that is intended to furnish pharmacological activity or other direct effect in the diagnosis, cure, mitigation, treatment, or prevention of disease, or to affect the structure or any function of the body of man or other animals. The term includes those components that may undergo chemical change in the manufacture of the drug product and are present in the drug product in a modified form intended to furnish the specified activity or effect.

In the present context, the term “hydrophilic” describes that something ‘likes water’, i.e. a hydrophilic molecule or portion of a molecule is one that typically is electrically polarized and capable of forming hydrogen bonds with water molecules, enabling it dissolve more readily in water than in oil or other “non-polar” solvents.

In the present context, the term “amphiphilic” describes a molecule (as a surfactant) having a polar water-soluble group attached to a water-insoluble hydrocarbon chain. Thus, one end of the molecule is hydrophilic (polar) and the other is hydrophobic (non-polar).

In the present context, the term “hydrophobic” denotes a compound tending to be electrically neutral and non-polar, and thus preferring other neutral and nonpolar solvents or molecular environments.

As used herein, the term “vehicle” means any solvent or carrier fluid in a pharmaceutical product that has no pharmacological role. For example, water is the vehicle for xilocaine and propylene glycol is the vehicle for many antibiotics.

In the present context, the term “solid dispersion” denotes a drug or active ingredient or substance dispersed on a particulate level in an inert vehicle, carrier, diluent or matrix in the solid state, i.e. usually a fine particulate dispersion.

In the present context, the term “solid solution” denotes a drug or active ingredient or substance dissolved on a molecular level in an inert vehicle, carrier, diluent or matrix in the solid state.

As used herein, the term “analogue” means a chemical compound that is structurally similar to another.

The term “drug” means a compound intended for use in diagnosis, cure, mitigation, treatment, or prevention of disease in man or other animals.

In this context, the term “dosage form” means the form in which the drug is delivered to the patient. This could be parenteral, topical, tablet, oral (liquid or dissolved powder), suppository, inhalation, transdermal, etc. In the present context oral also includes sublingual.

As used herein, the term “bioavailability” denotes the degree means to which a drug or other substance becomes available to the target tissue (blood stream) after administration.

As used herein, the term “bioequivalency” denotes a scientific basis on which generic and brand name drugs are compared with one another. For example, drugs are bioequivalent if they enter circulation at the same rate and to the same extent when given in similar doses under similar conditions. Parameters often used in bioequivalence studies are tₘₐₓ, cₘₐₓ, bioavailability measured as the area under the curve from a concentration/time curve measured from time of administration (Area Under Curve) and for a specified time, AUC₀₋ₜ or calculated beyond the measured time based on observed or known elimination to infinity which is to be understood as the time where no further absorption takes place (AUC_{0-infinity}). Other relevant parameters may be W₅₀, W₇₅ and/or MRT. Furthermore, in the present context, two compositions are regarded as bioequivalent if the value of the parameter used is within 80-125% of that of Prograf® or a similar commercially available tacrolimus-containing product used in the test.

In the present context “tₘₐₓ” denotes the time to reach the maximal plasma concentration (cₘₐₓ) after administration; AUC_{0-infinity} denotes the area under the plasma concentration versus time curve from time 0 to infinity; AUC₀₋ₜ denotes the area under the plasma concentration versus time curve from time 0 to time t; W₅₀ denotes the time where the plasma concentration is 50% or more of Cₘₐₓ; W₇₅ denotes the time where the plasma concentration is 75% or more of Cₘₐₓ; and MRT denotes mean residence time for tacrolimus (and/or an analogue thereof).

In this context, the term “medicine” means a compound used to treat disease, injury or pain. Medicine is justly distributed into “prophylactic,” i.e. the art of preserving health, and “therapeutic”, i.e. the art of restoring health.

In the present context, the terms “controlled release” and “modified release” are intended to be equivalent terms covering any type of release of tacrolimus from a composition of the invention that is appropriate to obtain a specific therapeutic or prophylactic response after administration to a subject. A person skilled in the art knows how controlled release/modified release differs from the release of plain tablets or capsules. The terms “release in a controlled manner” or “release in a modified manner” have the same meaning as stated above. The terms include slow release (that results in a lower Cₘₐₓ and later tₘₐₓ, but t_{1/2} is unchanged), extended release (that results in a lower Cₘₐₓ, later tₘₐₓ, but apparent t % is longer); delayed release (that result in an unchanged Cₘₐₓ, but lag time and, accordingly, tₘₐₓ is delayed, and t_{1/2} is unchanged) as well as pulsatile release, burst release, sustained release, prolonged release, chrono-optimized release, fast release (to obtain an enhanced onset of action) etc. Included in the terms is also e.g. utilization of specific conditions within the body e.g. different enzymes or pH changes in order to control the release of the drug substance.

In this context, the term “erosion” or “eroding” means a gradual breakdown of the surface of a material or structure, for example of a tablet or the coating of a tablet.

### CYP3A4 Inhibitors

It is contemplated that any known and pharmacologically acceptable compound capable of inhibiting cytochrome P450, i.e. inhibit CYP3A4 isoenzyme, is useful in the present invention.

Examples of useful CYP3A4 inhibitors are diethyl dithiocarbamate, ketoconazole, itraconazole, erythromycin, ritonavir, lanzoprazol, safrole, rutaecarpine, limonin, dipiperamide A (from white pepper), gomisin C (from schisandra fruit), paradisin A and paradisin B (from grape fruit juice).

Further useful compounds are those according to the following Formulae I-IV:

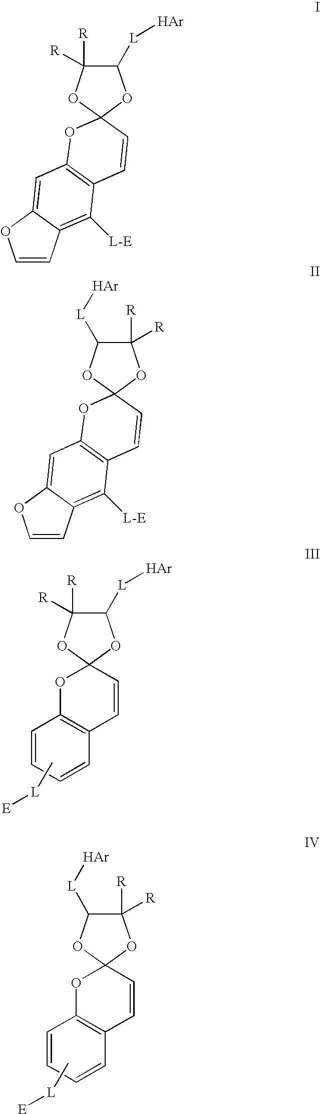

In each of the above structures, R is, independently, H or an optionally substituted C₁-C₁₅ alkyl group, L is an optionally substituted C₁-C₁₅ linear or branched, saturated, monounsaturated or polyunsaturated alkyl group optionally interrupted by one or plural nonadjacent sulfur or oxygen atoms and optionally terminated at one or both ends by oxygen, HAr is an optionally substituted C₆-C₂₄ aromatic group or heteroaromatic group optionally containing one or plural ring atoms selected from the group consisting of N, O, S, and P, and E is —OH, —COOH, —COOR, or an optionally substituted C₁-C₈ linear or branched, saturated, monounsaturated or polyunsaturated alkyl group optionally interrupted by one or plural nonadjacent oxygen or sulfur atoms, or E is a C₃-C₈ optionally substituted cyclic saturated, monounsaturated or polyunsaturated alkyl group optionally interrupted by one or plural nonadjacent oxygen or sulfur atoms, or E is optionally substituted HAr; and the optional substituents for the groups R, L, HAr and E are selected from the group consisting of a C₁-C₆ linear, branched or cyclic alkyl group, —OH, halogen, a C₁-C₅ alkoxy group, a C₁-C₅ alkyl carbonyloxy group and a C₁-C₅ alkoxycarbonyl group.

Preferably, the compounds of Formulae I-IV as well as those described below do not contain a peroxide (O—O) group. Disulfide groups (S—S) are not preferred, but may be present. Preferably E is an epoxide or dihydroxy radical such as —CH(OH)₂. E may also be an acid-opened epoxide group.

These compounds are unlimited with regard to stereochemistry, E-Z isomerism and all possibilities are included. Racemic mixtures are included as are each and every enantiomer and diasteriomer.

The groups R, L, HAr, and E may optionally be substituted with a C₁-C₆ linear, branched or cyclic alkyl group, —OH, a halogen atom, a C₁-C₅ alkoxy group, a C₁-C₅ alkyl carbonyloxy group, a C₁-C₅ alkoxycarbonyl group, etc. Such substituents also may be optionally substituted directly on the ring structures of Formulae I-IV regardless of whether such substituents appear on R, L, HAr or E.

Further useful compounds are:

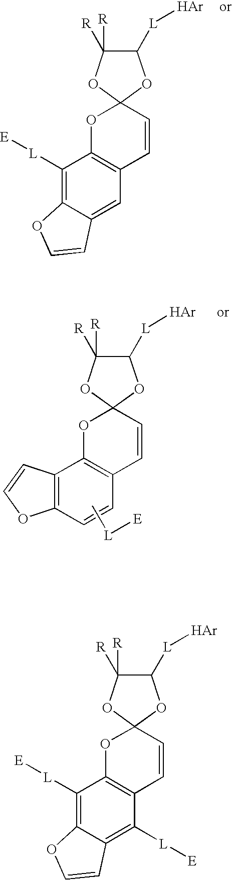

In each of the above structures, R is, independently, H or an optionally substituted C₁-C₁₅ alkyl group, L is an optionally substituted C₁-C₁₅ linear or branched, saturated, monounsaturated or polyunsaturated alkyl group optionally interrupted by one or plural nonadjacent sulfur or oxygen atoms and optionally terminated at one or both ends by oxygen, HAr is an optionally substituted C₆-C₂₄ aromatic group or heteroaromatic group optionally containing one or plural ring atoms selected from the group consisting of N, O, S, and P, and E is —OH, —COOH, —COOR, where R is defined above, or an optionally substituted C₁-C₈ linear or branched, saturated, monounsaturated or polyunsaturated alkyl group optionally interrupted by one or plural nonadjacent oxygen or sulfur atoms, or E is a C₃-C₈ optionally substituted cyclic saturated, monounsaturated or polyunsaturated alkyl group optionally interrupted by one or plural nonadjacent oxygen or sulfur atoms, or E is optionally substituted HAr; and the optional substituents for the groups R, L, HAr and E are selected from the group consisting of a C₁-C₆ linear, branched or cyclic alkyl group, —OH, halogen, a C₁-C₅ alkoxy group, a C₁-C₅ alkyl carbonyloxy group and a C₁-C₅ alkoxycarbonyl group.

Further useful compounds are compounds of f the formulae V-X:

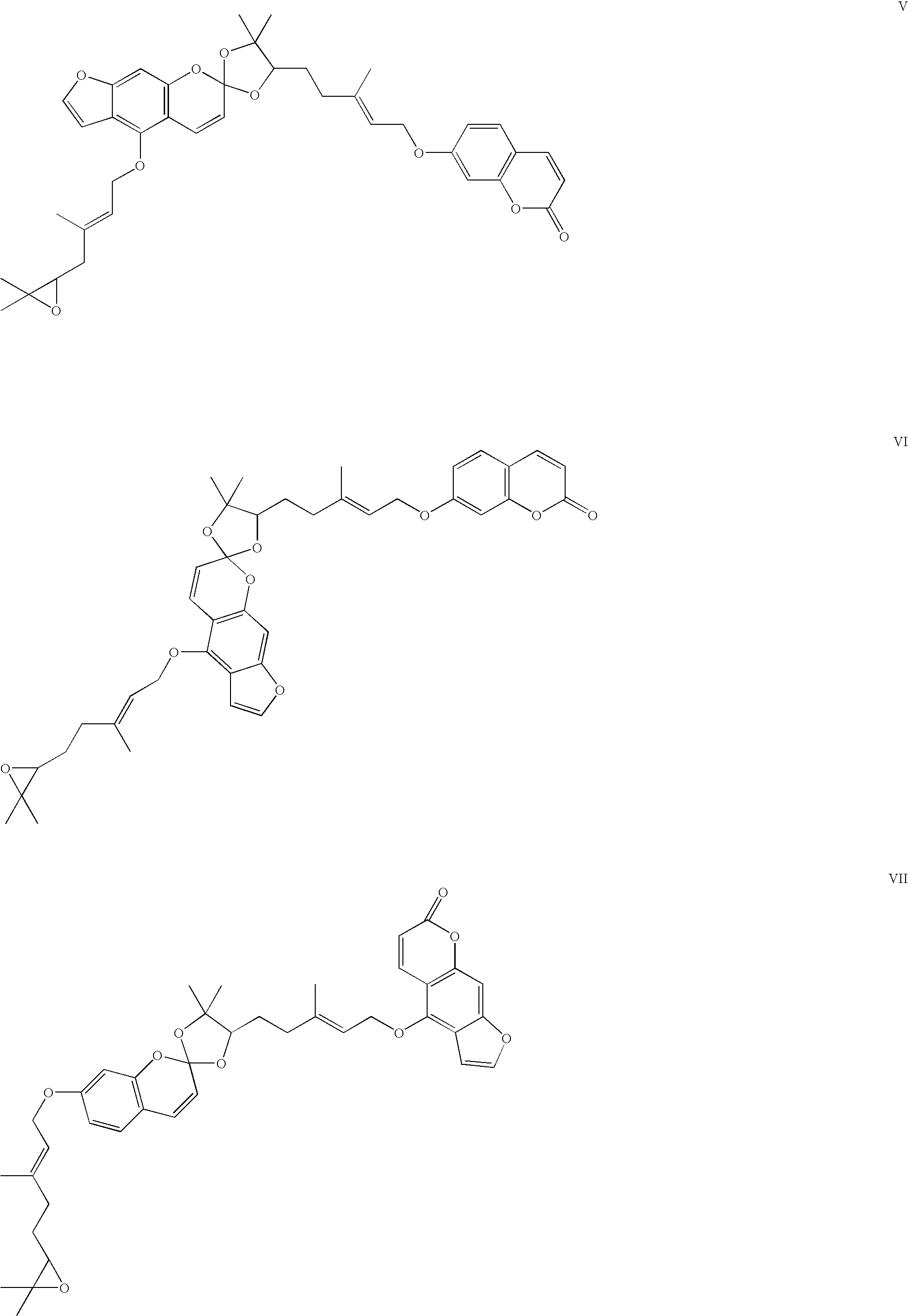

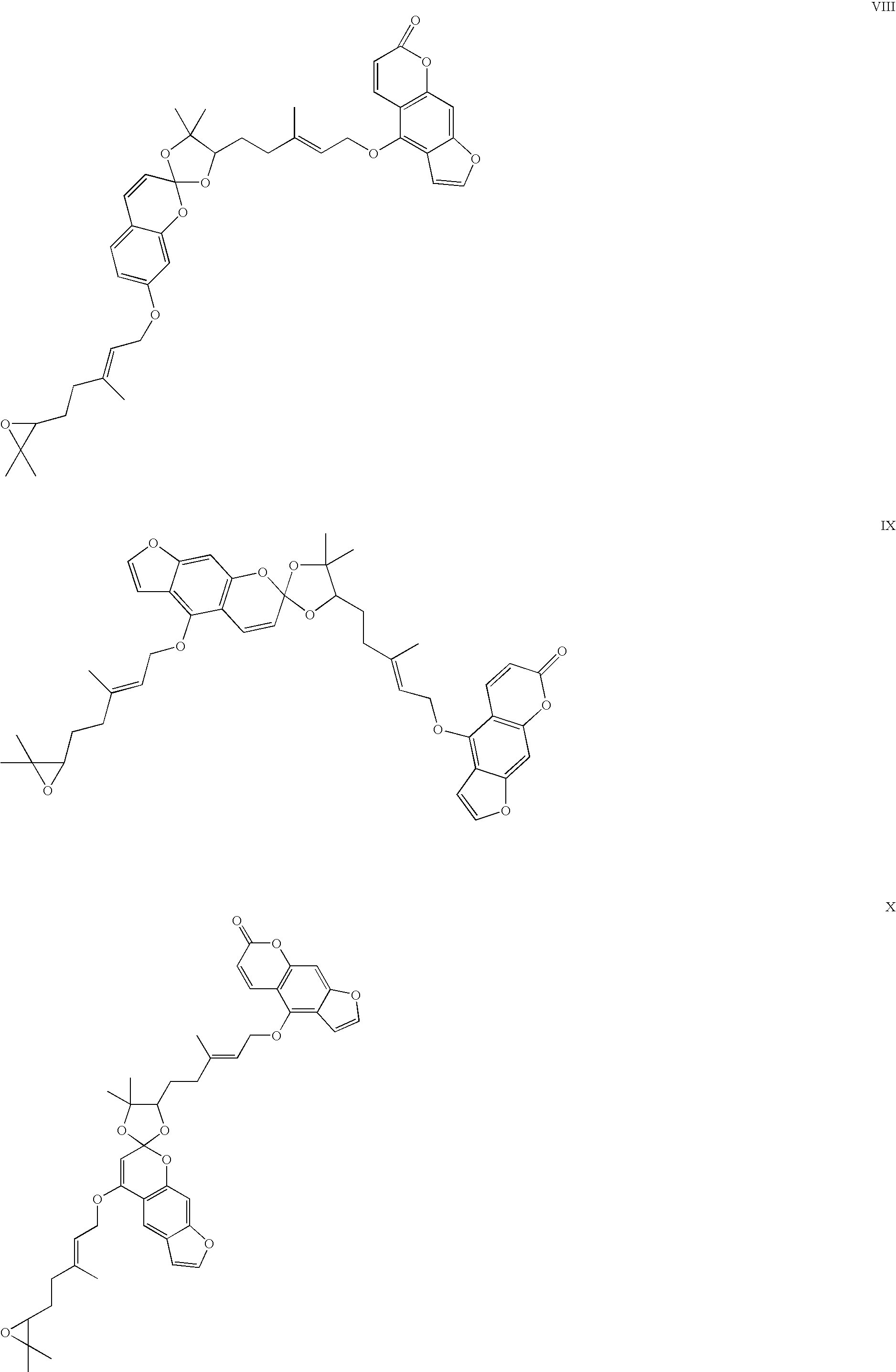

As noted above for Formulae I-IV, the compounds of Formulae V-X are unlimited with regard to stereochemistry, E-Z isomerism, etc.

The most preferred CYP3A4 inhibitor compounds are those of Formulae XI-XVI:

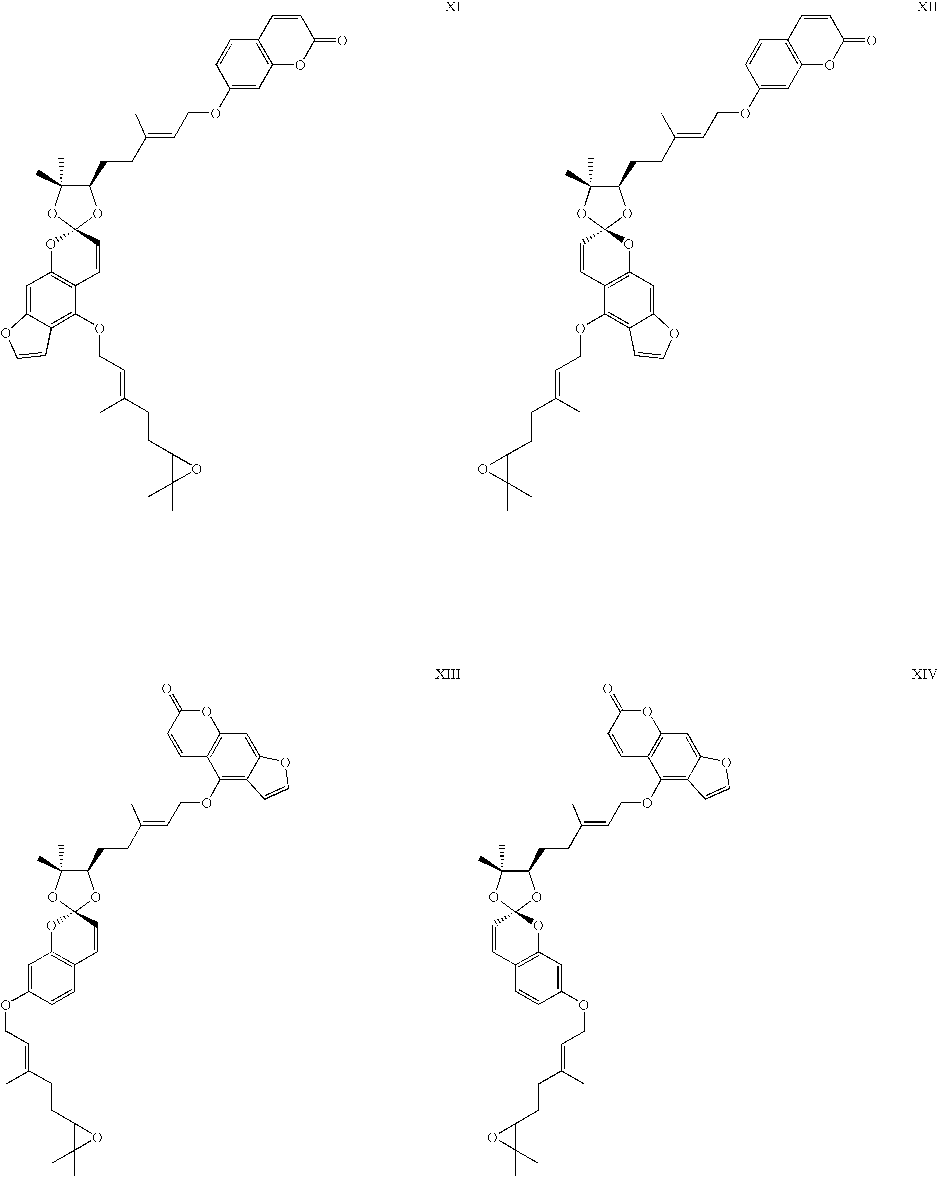

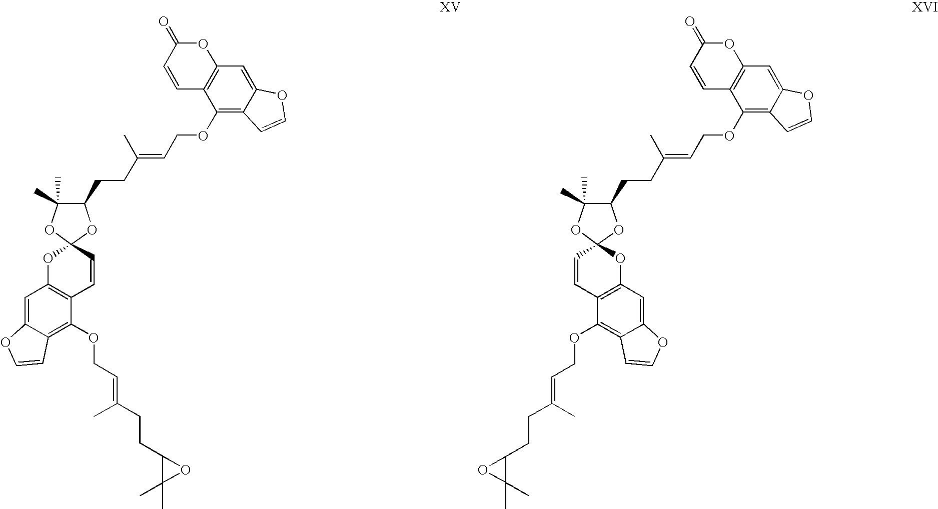

The compositions of the present invention contain at least one CYP3A4 inhibiting compound in an amount effective for enhancing the bioavailability of tacrolimus or an analogue thereof. It is to be understood that the composition of the invention may also, in addition to or as a substitute for the mentioned compounds of formulae V-XVI, comprise a citrus-derived extract, concentrate, peel, juice, oil, by-product, etc., (hereinafter referred to as the citrus-derived substance) and may be provided by any combination of these forms and may be derived from more than one citrus fruit. Useful citrus fruits herein include grapefruit, lemon, lime and, preferably, any citrus fruit naturally containing an invention first-pass effect inhibiting compound or mixture of such compounds.

It is contemplated that an effective amount of a CYP3A4 inhibitor is from about 2 mg to about 2000 mg, for example 2 mg, 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1500 mg.

### Tacrolimus

In a preferred embodiment of the present invention, the active ingredient tacrolimus or an analogue thereof is used in the form of a solid dispersion or a solid solution or a mixture of a solid dispersion and a solid solution. These forms are disclosed in the international publications WO2005/020993 and WO2005/020994. Accordingly, the active ingredient is dispersed or dissolved in a hydrophilic or water-miscible vehicle having a melting point (freezing point or pour point) of at least 20° C. in a concentration of between about 0.01 w/w % and about 15 w/w %, and which dispersion is forming a solid dispersion or solid solution at ambient temperature (room temperature).

Preferred vehicles in this respect is propylene glycol ester and deglycerol monolaureate including combinations hereof. The degree of estherification for the propylene glycol ester is at preferable at least 75%, such as at least 85% and preferable at least 95%.

The active ingredient is preferably tacrolimus or any analogue or derivative of tacrolimus, which exhibits either a pharmacological or a therapeutical activity, which is equivalent to that of tacrolimus (FK-506 or FR-900506). However, within the scope of the present invention is tacrolimus in any physical form (crystals, amorphous powder, any possible polymorphs, any possible solvates including the hydrate, anhydrate, complexes thereof etc.). Included is also any analogue, derivative or active metabolite of tacrolimus, pharmaceutically acceptable salts, solvates, complexes and prodrugs thereof.

The concentration of the active ingredient in the hydrophilic or water-miscible vehicle is at the most 15 w/w %, preferably at the most 10 w/w %, preferably at the most 8 w/w %, more preferably at the most 6 w/w %, even more preferably at the most 5 w/w %, at the most 4% w/w, especially at the most 3 w/w %, in particular at the most 2% w/w; and/or is at least about 0.05 w/w %, preferably at least about 0.1 w/w %, more preferably at least about 0.5 w/w %, especially at least about 0.7 w/w %, in particular at least about 1 w/w %.

Physically, the combination of active ingredient and vehicle may either form a solid dispersion, i.e. the active ingredient is dispersed in the vehicle in particulate form, or may form a solid solution, i.e. the active ingredient is dissolved in the vehicle at a molecular level. The active ingredient and the vehicle may also form a solid dispersion having therein a part of the active ingredient dissolved at a molecular level. The physical state of the dispersion and/or solution may be determined by using various techniques such as Hot Stage Microscopy (HSM), Differential Scanning Calorimetry (DSC), Scanning Electron Microscopy (SEM) optionally in combination with Energy Dispersive X-ray (EDX), and X-ray powder diffraction. In a preferred embodiment, the active ingredient is fully dissolved in the vehicle to form a solid solution at ambient temperature.

In a preferred embodiment, at least 80 w/w %, or at least 90 w/w %, or at least 99 w/w % of the tacrolimus is present in the composition as a solid dispersion or a solid solution in a hydrophilic or water-miscible vehicle.

Substrate for CYP3A4 and/or P-Glycoprotein, Cyclosporin A

The preferred substance being a substrate for CYP3A4 and/or P-glycoprotein is preferable a substrate for both and should preferable also have inhibiting properties and these may be by simple competition of the substrate on the sites thereby having an inhibitory effect. In a preferred embodiment of the invention, cyclosporine is used as the second substance.

In a preferred embodiment cyclosporine is dispersed or dissolved in a hydrophilic or water-miscible vehicle having a melting point (freezing point or pour point) of at least 20° C. in a concentration of between about 0.01 w/w % and about 15 w/w %, and which dispersion is forming a solid dispersion or solid solution at ambient temperature (room temperature).

In order to increase release of cyclosporine from the pharmaceutical composition and oral dosage form solubilization of cyclosporin is preferred, this include cosolvency, micellization, and complexation. Cosolvents (ethanol, propylene glycol, polyethylene glycol, tetrahydrofurfuryl alcohol polyethyleneglycol ether, and glycerin), surfactants (polyoxyethylene sorbitan monooleate [(Tween 80)], polyoxyethylene sorbitan monolaurate [(Tween 20)], and Cremophor EL), and cyclodextrins (α-cyclodextrin [(αCD)] and hydroxypropyl-β-cyclodextrin[(HPβCD)] may be used as solubilizing agents. Surfactants had a noticeable effect in increasing CsA solubility. Twenty percent solutions of Tween 20, Tween 80, and Cremophor EL increased the solubility by 60 to 160 fold. Cyclodextrins can increase the CsA solubility, but αCD is more effective than HPβCD.

Preferred vehicles in this respect is propylene glycol ester and deglycerol monolaureate including combinations hereof. The degree of estherification for the propylene glycol ester is at preferable at least 75%, such as at least 85% and preferable at least 95%.

### Pharmaceutical Composition

The pharmaceutical composition of the invention comprises tacrolimus, preferably in the form of a solid dispersion or solid solution, a substrate for CYP3A4 and/or P-glycoprotein and optionally one or more pharmaceutically acceptable excipients, for example one or more excipients useful as fillers, disintegrants, binders and/or lubricants.

Preferably, the pharmaceutical composition of the invention is in particulate form, for example in powder form. Preferably, the particulate material obtained is a free-flowing powder and therefore readily processable into e.g. solid dosage forms such as tablets, capsules or sachets. Normally, the particulate material has properties that are suitable in order to manufacture tablets by direct compression without addition of large amounts of further additives. A suitable test for testing the flowability of the particulate material is the method described in Ph.Eur. and measuring the flow rate of the material out of a funnel with a nozzle (orifice) diameter of 10.0 mm.

The particles may have a geometric weight mean diameter d_{gw} from about 10 μm to about 2000 μm, preferably from about 20 μm to about 2000 μm, more preferably from about 30 μm to about 2000 μm, more preferably from about 50 μm to about 2000 μm, more preferably from about 60 μm to about 2000 μm, more preferably from about 75 μm to about 2000 μm, more preferably from about 100 μm to about 1500 μm, more preferably from about 100 μm to about 1000 μm, more preferably from about 100 μm to about 700 μm, more preferably from about 50 μm to about 400 μm, more preferably from about 50 μm to about 350 μm, even more preferably from about 50 μm to about 300 μm, especially from about 50 μm to about 250 μm or, in particular, from about 100 μm to about 300 μm. In a preferred embodiment of the invention, the particles have a geometric weight mean diameter d_{gw} from about 50 μm to about 300 μm.

It is contemplated that the pharmaceutical composition of the invention, when administered to a human in need thereof, results in a relative AUCᵢₙᵥₑₙₜᵢₒₙ/AUCₛₑₚₐᵣₐₜₑ value of tacrolimus of at least about 1.1, the AUC values being determined under similar conditions and the AUCₛₑₚₐᵣₐₜₑ value is determined after administration of tacrolimus and CYP3A4 inhibitor in separate, however similar dosage forms as the combination product. In other words, there is observed at synergistic effect with respect to bioavailability of tacrolimus when administering the two active compounds simultaneously in the very same pharmaceutical formulation. Preferably, the relative AUCᵢₙᵥₑₙₜᵢₒₙ/AUCₛₑₚₐᵣₐₜₑ value is at least about 1.2, or at least about 1.3, or at least about 1.4, or at least about 1.5 or at least about 1.6.

It is to be understood that the pharmaceutical composition of the invention, when administered to a human in need thereof, also results in a relative AUCᵢₙᵥₑₙₜᵢₒₙ/AUC_{per se} value of tacrolimus of at least about 1.1, the AUC values being determined under similar conditions and the AUC_{per se} value is determined after administration of tacrolimus per se (i.e. without any administration of a compound having effect as CYP3A4 inhibitor). Preferably, the relative AUCᵢₙᵥₑₙₜᵢₒₙ/AUC_{per se} value is at least about 1.2, or at least about 1.3, or at least about 1.4, or at least about 1.5 or at least about 1.6. In a further aspect of the invention, an optimal ratio of the tacrolimus component and the CYP3A4 and P-glycoprotein substrate component is wherein the latter at one hand is present in an amount resulting in such low concentrations that possible undesired side effects are avoided and at the other hand the amount is sufficient to possess a substantial increase in bioavailability of tacrolimus, preferable above 10%, more preferred above 20% such as about 30% or more. Accordingly in one embodiment, the ratio of tacrolimus to cyclosporine of the dosage form and/or treatment according to the present invention is in a range of 1:0.1 to 1 to 200, preferable a ratio in the range of 1:0.5 to 1:100, such as in the range of 1:1 to 1:50, such as a ratio in range of 1:1.5 to 1:25 such as a ratio in the range of 1:2 to 1:20. The latter range has been demonstrated herein to be effective to increase bioavailability in the range of 30 to 100% compared to the administration of tacrolimus without CsA. However other ranges may be useful with other substrates than CsA depending on the binding kinetics of the substrate. In case this is lower than for CsA ratios with a relative higher amount compared to tacrolimus may be needed and vice versa for substrates with a substrate demonstrating higher stronger binding kinetics than CsA where lower doses may result in a substantial increase in tacrolimus bioavailability.

In a further aspect the invention relates to a stable composition, i.e. the substrate for CYP3A4 and/or P-glycoprotein and/or the tacrolimus or an analogue of tacrolimus is present in the composition in an amount of at least 90%, or at least 95%, or at least 100%, relative to the amount prior to storage, when assayed after at least 3 months of storage at a temperature of about 25° C. and a relative humidity of about 60%. In a preferred embodiment, at least 95 w/w % of each of the active compounds are present after 3 months of storage at a temperature of about 40° C. and a relative humidity of about 75%.

In a preferred embodiment of the invention, the substrate for CYP3A4 and/or P-glycoprotein is the active ingredient of an immediate release (IR) pharmaceutical formulation, i.e. release of the active ingredient from the pharmaceutical formulation begins immediately after administration to a patient; or the CYP3A4 inhibitor is the active ingredient of a modified release pharmaceutical formulation, for example a formulation which releases less than 20% w/w of the active ingredient during the first 5 hours after administration to a patient-corresponding to delaying the release of at least 80% w/w of the active ingredient for at least 5 hours after administration to the patient, that is to obtain a delayed therapeutic or prophylactic response.

Tacrolimus may be provided as the active ingredient of an immediate release (IR) pharmaceutical formulation or of an entero-coated immediate release formulation (IR-enteric), either co-formulated with the substrate for CYP3A4 and/or P-glycoprotein or formulated separately, i.e. in a separate formulation. Tacrolimus may also be provided as the active ingredient of a modified release pharmaceutical formulation, for example a formulation which releases less than 20% w/w of the active ingredient throughout the first 5 hours after administration to a patient (corresponding to delaying the release of at least 80% w/w of the active ingredient for at least 5 hours). The modified release formulation may be co-formulated, i.e. comprises tacrolimus as well as CYP3A4 inhibitor as active ingredients; or tacrolimus may be formulated separate from CYP3A4, i.e. in a separate formulation. The release of the substrate for CYP3A4 and/or P-glycoprotein may be in the IR form or may also be delayed, however it is considered an advantage that the substrate for CYP3A4 and/or P-glycoprotein, such as CsA is released substantial at the same time or just before the release of tacrolimus. In other words the release of tacrolimus may be simultaneously or delayed, such as delayed about 15 minutes, 30 minutes, 45 minutes, or event about an hour relative to the release of CsA from the dosage form.

Examples of useful formulations are provided in the examples herein.

### Dosage Forms

Useful dosage forms of the invention are solid oral dosage forms comprising the solid dispersion and/or solid solution and one or more pharmaceutically acceptable excipients, preferably unit dosage forms.

The pharmaceutical composition according to the invention is in particulate form and may be employed as such. However, in many cases it is more convenient to present the composition in the form of granules, pellets, microspheres, nanoparticles and the like or in the form of solid dosage forms including tablets, capsules and sachets and the like.

A solid dosage form according to the invention may be a single unit dosage form or it may in the form of a polydepot dosage form contain a multiplicity of individual units such as, e.g., pellets, beads and/or granules.

The dosage forms may further comprise pharmaceutically acceptable additives such as flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents and release modifying agents.

In a preferred embodiment, the dosage form comprises silica acid or a derivative or salt thereof including silicates, silicon dioxide and polymers thereof; and/or magnesium aluminosilicate and/or magnesium aluminometasilicate, bentonite, kaolin, magnesium trisilicate, montmorillonite and/or saponite. A particularly useful excipient to be included in the dosage forms is any silicon dioxide product having properties corresponding to Aeroperl® 300 (available from Degussa, Frankfurt, Germany).

A solid dosage form according to the present invention comprises a pharmaceutical composition in particulate form as described above. The details and particulars disclosed under this main aspect of the invention apply mutatis mutandis to the other aspects of the invention. Accordingly, the properties with respect to increase in bioavailability, changes in bioavailability parameters, reduction in adverse food effect as well as release of tacrolimus and/or an analogue thereof etc. described and/or claimed herein for pharmaceutical compositions in particulate form are analogues for a solid dosage form according to the present invention.

Normally, the concentration of the pharmaceutical composition in particulate form is in a range of from about 5 to 100% w/w such as, e.g., from about 10% to about 90% w/w, from about 15% to about 85% w/w, from about 20% to about 80% w/w, from about 25% to about 80% w/w, from about 30% to about 80% w/w, from about 35% to about 80% w/w, from about 40% to about 75% w/w, from about 45% to about 75% w/w or from about 50% to about 70% w/w of the dosage form. In an embodiment of the invention, the concentration of the pharmaceutical composition in particulate form is 50% w/w or more of the dosage form.

A solid dosage form according to the invention is obtained by processing the particulate material according to the invention by means of techniques well-known to a person skilled in the art. Normally, it involves further addition of one or more of the pharmaceutically acceptable excipients mentioned herein.

The composition or solid dosage form according to the invention may be designed to release tacrolimus and/or an analogue thereof in any suitable manner provided that the increase in bioavailability is present. Thus, the active substance may be released relatively fast in order to obtain an enhanced on-set of action, it may be released so as to follow zero or first order kinetics or it may be released in a modified manner in order to obtain a predetermined pattern of release. All of these ways are considered controlled manners. Plain formulations are also within the scope of the present invention.

The contemplated dosage recommendation for products of the present invention will be from 0.02 mg tacrolimus/kg/day to 0.15 mg tacrolimus/kg/day, dosed once a day.

In a preferred embodiment of the invention, the dosage form is provided as a single solid dosage form for oral administration comprising a first solid pharmaceutical composition containing tacrolimus or an analogue thereof as the active substance and second solid pharmaceutical composition containing a CYP3A4 inhibitor as the active substance, wherein the first and the second pharmaceutical composition are present in separate entities. By separating the active ingredients, any undesired interfering reactions and possible degradations are avoided, while the advantage of having a single unit dosage form is maintained.

Prior to combining the active ingredients into the single dosage form, each of these may be provided is in the form of granulate, granules, grains, beads or pellets which are optionally entero-coated or coated with a protective coating.

Preferably, the single solid dosage form is a tablet. Other useful forms are capsules and sachets.

In a tablet, the two active ingredients may be present in at least two separate layers, optionally separated by an intermediate, inactive layer. The tablet may also be prepared for example by direct compression of a granulate comprising tacrolimus, optionally entero-coated or coated with a protective coating, and a granulate comprising CYP3A4 inhibitor; or by direct compression of a granulate comprising both active ingredients.

### Vehicle

The hydrophilic or water-miscible vehicle to be used according to the invention, i.e. in the solid solution of tacrolimus, is preferably one having a melting point (freezing point or pour point) of at least 20° C., more preferably at least 30° C., more preferably at least 40° C., more preferably at least 50° C., even more preferably at least 52° C., even more preferably at least 55° C., even more preferably at least 59° C., especially at least 61° C., in particular at least 65° C.

Examples of useful hydrophilic or water-miscible vehicles to be used according to this invention are selected from the group consisting of polyethylene glycols, polyoxyethylene oxides, poloxamers, polyoxyethylene stearates, poly-epsilon caprolactone, polyglycolized glycerides such as Gelucire®, and mixtures thereof.

It is also contemplated that certain amphiphilic vehicles may be useful in the present invention, including those vehicles disclosed herein which may be amphiphilic in addition to being water-miscible.

In a preferred embodiment of the invention, the vehicle is a polyethylene glycol (PEG), in particular a PEG having an average molecular weight of at least 1500, preferably at least 3000, more preferably at least 4000, especially at least 6000. The polyethylene glycol may advantageously be mixed with one or more other hydrophilic or water-miscible vehicles, for example a poloxamer, preferably in a proportion (on a weight/weight basis) of between 1:3 and 10:1, preferably between 1:1 and 5:1, more preferably between and 3:2 4:1, especially between 2:1 and 3:1, in particular about 7:3. A specific example of a useful mixture is a mixture of PEG6000 and poloxamer 188 in the ratio 7:3.

For polyethylene glycols (PEG), the melting point (freezing point or pour point) increases as the average molecular weight increases. For example, PEG 400 is in the range of 4-8° C., PEG 600 is in the range of 20-25° C., PEG1500 is in the range of 44-48° C., PEG2000 is about 52° C., PEG 4000 is about 59° C., PEG 6000 is about 65° C. and PEG 8000 is about 61° C.

Useful poloxamers (also denoted polyoxypropylene-polyoxyethylene block copolymers) are for example poloxamer 188, poloxamer 237, poloxamer 338 or poloxamer 407 or other block copolymers of ethylene oxide and propylene oxide such as the Pluronic® and/or Tetronic® series. Suitable block copolymers of the Pluronic® series include polymers having a molecular weight of about 3,000 or more such as, e.g. from about 4,000 to about 20,000 and/or a viscosity (Brookfield) from about 200 to about 4,000 cps such as, e.g., from about 250 to about 3,000 cps. Suitable examples include Pluronic® F38, P65, P68LF, P75, F77, P84, P85, F87, F88, F98, P103, P104, P105, F108, P123, F123, F127, 10R8, 17R8, 25R5, 25R8 etc. Suitable block copolymers of the Tetronic® series include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60° C. for substances that are pastes at room temperature and at 77° C. for substances that are solids at room temperature.

In a preferred embodiment of the present invention, the poloxamer is poloxamer 188, which has an average molecular weight of about 8400 and a melting point of about 50-54° C.

Other useful hydrophilic or water-miscible vehicles may be polyvinylpyrrolidones, polyvinyl-polyvinylacetate copolymers (PVP-PVA), polyvinyl alcohol (PVA), polymethacrylic polymers (Eudragit RS; Eudragit RL, Eudragit NE, Eudragit E), cellulose derivatives including hydroxypropyl methylcellulose (HPMC), hydroxypropyl cellulose (HPC), methylcellulose, sodium carboxymethylcellulose, hydroxyethyl cellulose, pectins, cyclodextrins, galactomannans, alginates, carragenates, xanthan gums and mixtures thereof.

“Polyglycolized glycerides” denotes a mixture of mono-, di- and triglycerides and polyethylene glycol (PEG) mono- and diesters, preferably of molecular weight between 200 and 600, where appropriate of free glycerol and free PEG, whose HLB value is adjusted by the length of the PEG chain, and whose melting point is adjusted by the length of the chains of the fatty acids, of the PEG and by the degree of saturation of the fatty chains, and hence of the starting oil; examples of such mixtures are Gelucire®. Gelucire® compositions are inert semi-solid waxy materials which are amphiphilic in character and are available with varying physical characteristics. They are surface active in nature and disperse or solubilize in aqueous media forming micelles, microscopic globules or vesicles. They are identified by their melting point/HLB value. The melting point is expressed in degrees Celsius and the HLB (Hydrophile-Lipophile Balance) is a numerical scale extending from 0 to approximately 20. Lower HLB values denote more lipophilic and hydrophobic substances, and higher values denote more hydrophilic and lipophobic substances. The affinity of a compound for water or for oily substances is determined and its HLB value is assigned experimentally. One or a mixture of different grades of Gelucire® excipient may be chosen to achieve the desired characteristics of melting point and/or HLB value. They are mixtures of monoesters, diesters and/or triesters of glycerides of long chain (C₁₂ to C₁₈) fatty acids, and PEG (mono- and/or di) esters of long chain (C₁₂ to C₁₈) fatty acids and can include free PEG. Gelucire® compositions are generally described as fatty acid esters of glycerol and PEG esters or as polyglycolized glycerides. Gelucire® compositions are characterized by a wide range of melting points of from about 33° C. to about 64° C. and most commonly from about 35° C. to about 55° C., and by a variety of HLB values of from about 1 to about 14, most commonly from about 7 to about 14. For example, Gelucire® 50/13 designates a melting point of approximately 50° C. and an HLB value of about 13 to this grade of Gelucire®.

### Pharmaceutically Acceptable Excipients

Examples of suitable excipients for use in a composition or solid dosage form according to the present invention include fillers, diluents, disintegrants, binders, lubricants and the like and mixtures thereof. As the composition or solid dosage form according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents and the like.

Examples of suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in a composition or solid dosage form of the invention are e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other additives in a composition or a solid dosage form according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehyde sulfoxylate, sodium metabisulfite, sodium thiosulfate, sulfur dioxide, tocopherol, tocopherol acetate, tocopherol hemisuccinate, TPGS or other tocopherol derivatives, etc. The carrier composition may also contain e.g. stabilising agents. The concentration of an antioxidant and/or a stabilizing agent in the carrier composition is normally from about 0.1% w/w to about 5% w/w.

A composition or solid dosage form according to the invention may also include one or more surfactants or substances having surface-active properties. It is contemplated that such substances are involved in the wetting of the slightly soluble active substance and thus, contributes to improved solubility characteristics of the active substance.

Suitable excipients for use in a composition or a solid dosage form according to the invention are surfactants such as, e.g., amphiphillic surfactants as those disclosed in WO 00/50007 in the name of Lipocine, Inc. Examples of suitable surfactants are
i) polyethoxylated fatty acids such as, e.g. fatty acid mono- or diesters of polyethylene glycol or mixtures thereof such as, e.g. mono- or diesters of polyethylene glycol with lauric acid, oleic acid, stearic acid, myristic acid, ricinoleic acid, and the polyethylene glycol may be selected from PEG 4, PEG 5, PEG 6, PEG 7, PEG 8, PEG 9, PEG 10, PEG 12, PEG 15, PEG 20, PEG 25, PEG 30, PEG 32, PEG 40, PEG 45, PEG 50, PEG 55, PEG 100, PEG 200, PEG 400, PEG 600, PEG 800, PEG 1000, PEG 2000, PEG 3000, PEG 4000, PEG 5000, PEG 6000, PEG 7000, PEG 8000, PEG 9000, PEG 1000, PEG 10,000, PEG 15,000, PEG 20,000, PEG 35,000,
ii) polyethylene glycol glycerol fatty acid esters, i.e. esters like the above-mentioned but in the form of glyceryl esters of the individual fatty acids;
iii) glycerol, propylene glycol, ethylene glycol, PEG or sorbitol esters with e.g. vegetable oils like e.g. hydrogenated castor oil, almond oil, palm kernel oil, castor oil, apricot kernel oil, olive oil, peanut oil, hydrogenated palm kernel oil and the like,
iv) polyglycerized fatty acids like e.g. polyglycerol stearate, polyglycerol oleate, polyglycerol ricinoleate, polyglycerol linoleate,
v) propylene glycol fatty acid esters such as, e.g. propylene glycol monolaurate, propylene glycol ricinoleate and the like,
vi) mono- and diglycerides like e.g. glyceryl monooleate, glyceryl dioleae, glyceryl mono- and/or dioleate, glyceryl caprylate, glyceryl caprate etc.; vii) sterol and sterol derivatives;
viii) polyethylene glycol sorbitan fatty acid esters (PEG-sorbitan fatty acid esters) such as esters of PEG with the various molecular weights indicated above, and the various Tween® series;
ix) polyethylene glycol alkyl ethers such as, e.g. PEG oleyl ether and PEG lauryl ether;
x) sugar esters like e.g. sucrose monopalmitate and sucrose monolaurate;
xi) polyethylene glycol alkyl phenols like e.g. the Triton® X or N series;
xii) polyoxyethylene-polyoxypropylene block copolymers such as, e.g., the Pluronic® series, the Synperonic® series, Emkalyx®, Lutrol®, Supronic® etc. The generic term for these polymers is “poloxamers” and relevant examples in the present context are Poloxamer 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407;
xiii) sorbitan fatty acid esters like the Span® series or Ariacel® series such as, e.g. sorbinan monolaurate, sorbitan monopalmitate, sorbitan monooleate, sorbitan monostearate etc.;
xiv) lower alcohol fatty acid esters like e.g. oleate, isopropyl myristate, isopropyl palmitate etc.;
xv) ionic surfactants including cationic, anionic and zwitterionic surfactants such as, e.g. fatty acid salts, bile salts, phospholipids, phosphoric acid esters, carboxylates, sulfates and sulfonates etc.

When a surfactant or a mixture of surfactants is present in a composition or a solid dosage form of the invention, the concentration of the surfactant(s) is normally in a range of from about 0, 1-80% w/w such as, e.g., from about 0.1 to about 20% w/w, from about 0.1 to about 15% w/w, from about 0.5 to about 10% w/w, or alternatively, from about 0.10 to about 80% w/w such as, e.g. from about 10 to about 70% w/w, from about 20 to about 60% w/w or from about 30 to about 50% w/w.

### Release Profiles

In the present context “tₘₐₓ” denotes the time to reach the maximal plasma concentration (cₘₐₓ) after administration.

Thus, the dosage form of the invention may further comprise one or more release modifying agents selected from the group consisting of water-miscible polymers, water-insoluble polymers, oils and oily materials.

The water-insoluble polymer may be ethyl cellulose, cellulose acetate, cellulose nitrate, and mixtures thereof. The water-miscible polymer may also be a cellulose derivative selected from the group consisting of hydroxypropyl methylcellulose (HPMC), hydroxypropyl cellulose (HPC), methylcellulose, sodium carboxymethylcellulose, hydroxyethyl cellulose, poloxamers, polyoxyethylene stearates, poly-ε-caprolactone, polyvinylpyrrolidone (PVP), polyvinylpyrrolidone-polyvinylacetate copolymer PVP-PVA, polymethacrylic polymers and polyvinyl alcohol (PVA), poly(ethylene oxide) (PEO) and mixtures thereof. Examples of especially useful polymethacrylic polymers are Eudragit® RS, Eudragit® RL, Eudragit® NE and Eudragit® E.

The oil or oily material may be hydrophilic and hydrophobic oils or oily materials.

Hydrophilic oil or oily material may be polyether glycols such as polypropylene glycols; polyoxyethylenes; polyoxypropylenes; poloxamers; polyglycolized glycerides such as Gelucire®, for example Gelucire® 50/13, Gelucire® 44/14, Gelucire® 50/10, Gelucire® 62/05 and mixtures thereof.

Hydrophobic oil or oily material may have a melting point of at least about 20° C. Useful examples are straight chain saturated hydrocarbons, sorbitan esters, paraffins; fats and oils such as cacao butter, beef tallow, lard, polyether glycol esters; higher fatty acid such as stearic acid, myristic acid, palmitic acid, higher alcohols such as cetanol, stearyl alcohol, low melting point waxes such as glyceryl monostearate, glyceryl monooleate, hydrogenated tallow, myristyl alcohol, stearyl alcohol, substituted and/or unsubstituted monoglycerides, substituted and/or unsubstituted diglycerides, substituted and/or unsubstituted triglycerides, yellow beeswax, white beeswax, carnauba wax, castor wax, japan wax, acetylate monoglycerides; NVP polymers, PVP polymers, acrylic polymers, and mixtures thereof.

The oil or oily-like material may also be a sorbitan ester such as, e.g., sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof.

The oil or oily-like material may of course comprise a mixture of different oils or oily-like materials such as, e.g., a mixture of hydrophilic and/or hydrophobic materials.

Other suitable oils or oily-like materials may be solvents or semi-solid excipients like, e.g. propylene glycol, polyglycolised glycerides including Gelucire 44/14, complex fatty materials of plant origin including theobroma oil, carnauba wax, vegetable oils like e.g. almond oil, coconut oil, corn oil, cottonseed oil, sesame oil, soya oil, olive oil, castor oil, palm kernels oil, peanut oil, rape oil, grape seed oil etc., hydrogenated vegetable oils such as, e.g. hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid interesterified semi-synthetic glycerides including Miglycol 810/812; amide or fatty acid alcolamides including stearamide ethanol, diethanolamide of fatty coconut acids, acetic acid esters of mono and di-glycerides, citric acid esters of mono and di-glycerides, lactic acid esters of mono and diglycerides, mono and di-glycerides, poly-glycerol esters of fatty acids, poly-glycerol poly-ricinoleate, propylene glycol esters of fatty acids, sorbitan monostearates, sorbitan tristearates, sodium stearoyl lactylates, calcium stearoyl lactylates, diacetyl tartaric acid esters of mono and di-glycerides etc.

A delayed release of active ingredient is desired in order to increase the bioavailability of active ingredient by delivering the ingredient in the gastrointestinal tract, i.e. the release predominantly takes place after passage of the stomach. For example, the dosage form of the present invention may be designed in order to release, after oral administration to a mammal in need thereof, at the most about 10 w/w %, preferably at the most about 7.5 w/w %, more preferably at the most about 5 w/w %, especially at the most about 2 w/w % of the total amount of active ingredient within the first 3 hours, preferably within 2 hours, more preferably within 1 hours, in particular within about 30 minutes after administration.

Further, the solid dosage form of the invention may, upon oral administration to a mammal in need thereof, release at least about 50 w/w % of the active ingredient within 24 hours, preferably within about 20 hours, more preferably within about 18 hours, especially within about 15 hours, in particular within about 12 hours.

Delayed release is mainly brought about by some kind of enteric coating. Whereas semipermeable coating will show some kind of delayed release, it may not preciously enough “delay” release. Additionally it requires a certain amount of time to release the content. The coating sought for this invention, is a pH dependant coating. This type of coating is very resistant to release of drug until a certain pH is reached. Within a small increment in the pH value, i.e. within an increase in pH of about 0.2 to 0.4, the film alters properties and becomes permeable.

Accordingly, the solid dosage forms of the invention may exhibit a delayed release of active ingredient by means of an enteric coating using a water-miscible polymer having a pH-dependant solubility in water. Examples of pH-sensitive polymers, which are relatively insoluble and impermeable at the pH of the stomach, but which are more soluble and permeable at the pH of the small intestine and colon include, but are not limited to, polyacrylamides; phthalate derivatives such as acid phthalates of carbohydrates including amylose acetate phthalate, cellulose acetate phthalate, cellulose acetate terephthalate, cellulose acetate isophthalate, other cellulose ester phthalates, cellulose ether phthalates, hydroxypropyl cellulose phthalate, hydroxypropylcellulose acetate phthalate, hydroxypropyl ethylcellulose phthalate, hydroxypropyl methylcellulose phthalate (HMPCP), methylcellulose phthalate, methyl cellulose acetate phthalate, polyvinyl acetate phthalate, polyvinyl acetate hydrogen phthalate, sodium cellulose acetate phthalate, starch acid phthalate; phthalates of other compounds including polyvinyl acetate phthalate (PVAP); other cellulose derivatives including hydroxypropyl methylcellulose acetate succinate (HPMCAS), carboxymethylcellulose, cellulose acetate trimellitate; alginates; carbomers; polyacrylic acid derivatives such as acrylic acid and acrylic ester copolymers, polymethacrylic acid and esters thereof, poly acrylic methacrylic acid copolymers, methacrylic acid copolymers (for example Eudragit® L and Eudragit® S); styrene-maleic acid dibutyl phthalate copolymer, styrene-maleic acid polyvinylacetate phthalate copolymer, styrene and maleic acid copolymers; shellac, starch glycolate; polacrylin; vinyl acetate and crotonic acid copolymers and mixtures thereof. pH-sensitive polymers of specific interest include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

A first delayed release embodiment according to the invention is a “pH-dependent coated dosage form” such as, e.g., a tablet or a capsule. In the case of a tablet it comprises a tablet core comprising tacrolimus e.g. in a solid solution/dispersion as a multiparticulate product, a controlled release matrix of e.g. HPMC (hypromellose), a disintegrant, a lubricant, and one or more pharmaceutical carriers, such core being coated with a material, preferably a polymer, which is substantially insoluble and impermeable at the pH of the stomach, and which is more soluble and permeable at the pH of the small intestine. Preferably, the coating polymer is substantially insoluble and impermeable at pH<5.0, and water-soluble at pH>5.0. The tablet core may be coated with an amount of polymer sufficient to assure that substantially no release of tacrolimus from the dosage form occurs until the dosage form has exited the stomach and has resided in the small intestine for about 15 minutes or greater, preferably about 30 minutes or greater, thus assuring that minimal tacrolimus is released in the duodenum. Mixtures of a pH-sensitive polymer with a water-insoluble polymer may also be employed. Tablets are coated with an amount of polymer comprising from about 10% to about 80% of the weight of the tacrolimus-containing tablet core. Preferred tablets are coated with an amount of polymer comprising about 15% to about 50% of the weight of the tacrolimus tablet core.

pH-sensitive polymers which are very insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

The delay time before release of tacrolimus, after the “pH-dependent coated tablet” dosage form has exited the stomach, may be controlled by choice of the relative amounts of Eudragit-L® and Eudragit-S® in the coating, and by choice of the coating thickness. Eudragit-L® films dissolve above pH 6.0, and Eudragit-S® films dissolve above 7.0, and mixtures dissolve at an intermediate pH. Since the pH of the duodenum is approximately 6.0 and the pH of the colon is approximately. 7.0, coatings composed of mixtures of Eudragit-L® and Eudragit-S® provide protection of the duodenum from tacrolimus. If it is desired to delay release of tacrolimus until the tacrolimus-containing “pH-dependent coated tablet” has reached the colon, Eudragit-S® may be used as the coating material, as described by Dew et al. (Br. J. Clin. Pharmac. 14 (1982) 405-408). In order to delay the release of tacrolimus for about 15 minutes or more, preferably 30 minutes or more, after the dosage form has exited the stomach, preferred coatings comprise from about 9:1 to about 1:9 Eudragit-L®/Eudragit-S®, more preferably from about 9:1 to about 1:4 Eudragit-L®/Eudragit-S®. The coating may comprise from about 3% to about 70% of the weight of the uncoated tablet core. Preferably, the coating comprises from about 5% to about 50% of the weight of the tablet core.

The release of the active substance from a composition having a delayed release coating could also be an enzymatic reaction, if for example Zein or mono/di-glyceride mixtures are employed as coating material.

### Materials and Methods

### Materials

Tacrolimus (supplied by Eurotrade); batch no RD 03-111
Lactose monohydrate 200 mesh or 125 mesh (from DMV)
Polyethylene glycol 6000, Pluracol® E6000 (from BASF)
Poloxamer 188, Pluronic® F-68 (from BASF)
Glyceryl monostearate, Rylo® MD50, (from Danisco Cultor), Ph.Eur.; batch no. 4010056276
Magnesium stearate
Croscarmellose sodium, Ac-Di-Sol® (from FMC)
Eudragit® L30D.55 (from Degussa)
Triethyl citrate (from Merck)
Anti-foam emulsion (from Unikem)
Micro talc

### Ketonazole

### Limonin

Spiro ortho esters prepared as disclosed in WO99/09976 (compounds V-XIV) and WO2004/037827
HPMC/hypromellose refers to Metolose 90SH (type 2910, 2208—for example 15,000 cP) or
Metolose 60SH (type 2910) from ShinEtsu available in various degrees of polymerization (viscosity 3-100,000 cP).

Tablets, capsules or granules might be enteric coated with different types of polymers such as hydroxypropylmethylcellulose acetate succinate (Aqoat), cellulose acetate phthalate CAP, hydroxypropylmethylcellulose phthalate HPMCP or methacrylic acid copolymers such as Eudragit L30D, Eudragit 100/S, Eudragit 100/L.

### Methods

### Determination of Weight Variation

The tablets prepared in the Examples herein were subjected to a test for weight variation performed in accordance with Ph. Eur.

### Determination of Average Tablet Hardness

The tablets prepared in the Examples herein were subjected to at test for tablet hardness employing Schleuniger Model 6D apparatus and performed in accordance with the general instructions for the apparatus.

### Determination of Disintegration Time

The time for a tablet to disintegrate, i.e. to decompose into particles or agglomerates, was determined in accordance with Ph. Eur.

Determination of Geometric Weight Mean Diameter d_{gw}

The geometric weight mean diameter was determined by employment of a method of laser diffraction dispersing the particulate material obtained (or the starting material) in air. The measurements were performed at 1 bar dispersive pressure in Sympatec Helos equipment, which records the distribution of the equivalent spherical diameter. This distribution is fitted to a log normal volume-size distribution.

When used herein, “geometric weight mean diameter” means the mean diameter of the log normal volume-size distribution.

### In Vitro Dissolution Tests

The following test methods were applies to the compositions and dosage forms of the present invention.

### Test 1:

In vitro dissolution test according to USP Method A, delayed release articles (USP paddle method; rotation speed: 50 rpm; 37° C.; after 2 hours in acidic medium, the medium is changed to phosphate buffer pH 6.8.).

### Test 2:

In vitro dissolution test in aqueous dissolution medium adjusted to pH 4.5 (900 ml water with 0.005% HPC (hydroxypropylcellulose) adjusted to pH4.5; 37° C.; USP Paddle method; rotation speed: 50 rpm).

As explained above the individual active substances of the present invention may be formulated in individual matrix compositions which may be combined into final pharmaceutical products or dosage units. Accordingly, the examples include such individual matrix compositions as well as combined compostions.

The following non-limiting examples illustrate the invention.

### EXAMPLE 1

### Immediate Release Composition of Tacrolimus and a CYP3A4 Inhibitor Compound

The following tablet compositions may be prepared

| Substance | | mg | |
|---|---|---|---|
| Tacrolimus | Active ingredient | 1.0 | |
| | CYP3A4 and P- | | |
| | glycoprotein | | |
| Cyclosporin A | substrate | 1.0 | |
| Lactose 200 mesh | Carrier | 42.7 | |
| PEG 6000 | Vehicle | 34.3 | |
| Poloxamer 188 | Vehicle | 14.7 | |
| Croscarmellose sodium (Ac-di-sol) | Filler | 4.9 | |
| Dimeticone 350 | Antioxidant | 0.25 | microgram |
| Citric acid monohydrate | Antioxidant | 25 | microgram |
| Isopropyl alcohol | Antioxidant | 0.5 | microL |
| Butyl hydroxy toluene (BHT) | Antioxidant | 5 | microgram |
| Total | | 100.0 | |

The following tablet compositions were prepared:

### Composition 1A:

| Substance | | Mg | |
|---|---|---|---|
| Tacrolimus | Active ingredient | 1.0 | |
| Spiro ortho ester | CYP3A4 inhibitor | 2.0 | |
| Lactose 200 mesh | Carrier | 41.7 | |
| PEG 6000 | Vehicle | 34.3 | |
| Poloxamer 188 | Vehicle | 14.7 | |
| Croscarmellose sodium (Ac-di-sol) | Filler | 4.9 | |
| Dimeticone 350 | Antioxidant | 0.25 | microgram |
| Citric acid monohydrate | Antioxidant | 25 | microgram |
| Isopropyl alcohol | Antioxidant | 0.5 | microL |
| Butyl hydroxy toluene (BHT) | Antioxidant | 5 | microgram |
| Total | | 100.0 | |

### Composition 1B:

| Substance | | Mg | |
|---|---|---|---|
| Tacrolimus | Active ingredient | 1.0 | |
| Limonin | CYP3A4 inhibitor | 5.0 | |
| Lactose 200 mesh | Carrier | 41.7 | |
| PEG 6000 | Vehicle | 34.3 | |
| Poloxamer 188 | Vehicle | 14.7 | |
| Croscarmellose sodium (Ac-di-sol) | Filler | 4.9 | |
| Dimeticone 350 | Antioxidant | 0.25 | microgram |
| Citric acid monohydrate | Antioxidant | 25 | microgram |
| Isopropyl alcohol | Antioxidant | 0.5 | microL |
| Butyl hydroxy toluene (BHT) | Antioxidant | 5 | microgram |
| Total | | 103.0 | |

### Composition 1C:

| Substance | | Mg | |
|---|---|---|---|
| Tacrolimus | Active ingredient | 1.0 | |
| Ketoconazole | CYP3A4 inhibitor | 200.0 | |
| Lactose 200 mesh | Carrier | 41.7 | |
| PEG 6000 | Vehicle | 34.3 | |
| Poloxamer 188 | Vehicle | 14.7 | |
| Croscarmellose sodium (Ac-di-sol) | Filler | 4.9 | |
| Dimeticone 350 | Antioxidant | 0.25 | microgram |
| Citric acid monohydrate | Antioxidant | 25 | microgram |
| Isopropyl alcohol | Antioxidant | 0.5 | microL |
| Butyl hydroxy toluene (BHT) | Antioxidant | 5 | microgram |
| Total | | 298.0 | |

Film coating:

The tablets of examples 1A, 1B, 1C and 1D may subsequently be coated with the following film coating:

| Ingredients | | mg/tablet |
|---|---|---|
| Eudragit ® L30D | Coating agent | 42.4 |
| Purified water | Dispersion medium | 58.2 |
| Triethyl acetylcitrate | Plasticizer | 1.9 |
| Dow Corning 1510 | Anti-foam emulsion | 0.2 |
| Talc | Anti-caking agent | 3.2 |
| Total | | 105.9 |

The coating suspension was prepared by mixing triethyl acetylcitrate, antifoam emulsion and purified water in Ultra Turrax apparatus at 9500 rpm for 30 min. After 1 minute talc was added. The mixture was passed through sieve no. 300 and stirred by a magnet stirrer. Eudragit was passed through sieve no. 300 and added the mixture, which was stirred for 5 minutes.

In a preferred embodiment, cyclosporin may be present in a coating in an amount of 2, 5 or optionally 10 mg in a coating, such as in the following and prepared in the same manner as in Example 3.

| Ingredients | | mg/tablet |
|---|---|---|
| Eudragit ® L30D | Coating agent | 41.4 |
| Cyclosporine A | CYP3A4 substrate | 5.0 |
| Purified water | Dispersion medium | 56.2 |
| Triethyl acetylcitrate | Plasticizer | 1.9 |
| Dow Corning 1510 | Anti-foam emulsion | 0.2 |
| Talc | Anti-caking agent | 3.2 |

### EXAMPLE 2

### Tacrolimus Tablet Film-Coated with a CP3A4 Inhibitor Compound

The following tablet composition may be prepared:

| Substance | | Mg | |
|---|---|---|---|
| Tacrolimus | Active ingredient | 2.0 | |
| Lactose 200 mesh | Carrier | 41.7 | |
| PEG 6000 | Vehicle | 34.3 | |
| Poloxamer 188 | Vehicle | 14.7 | |
| Croscarmellose sodium (Ac-di-sol) | Filler | 4.9 | |
| Dimeticone 350 | Antioxidant | 0.25 | microgram |
| Citric acid monohydrate | Antioxidant | 25 | microgram |
| Isopropyl alcohol | Antioxidant | 0.5 | microL |
| Butyl hydroxy toluene (BHT) | Antioxidant | 5 | microgram |
| Total | | 101.0 | |

### Film Coating:

The tablet is subsequently coated with the following film coating:

| Ingredients | | mg/tablet |
|---|---|---|
| Eudragit ® L30D | Coating agent | 42.4 |
| Spiro ortho ester | CYP3A4 inhibitor | 2.0 |
| Purified water | Dispersion medium | 58.2 |
| Triethyl acetylcitrate | Plasticizer | 1.9 |
| Dow Corning 1510 | Anti-foam emulsion | 0.2 |
| Talc | Anti-caking agent | 3.2 |
| Total | | 107.9 |

The coating suspension is prepared by mixing triethyl acetylcitrate, spiro ortho ester, antifoam emulsion and purified water in Ultra Turrax apparatus at 9500 rpm for 30 min. After 1 minute talc is added. The mixture is passed through sieve no. 300 and stirred by a magnet stirrer. Eudragit is passed through sieve no. 300 and added the mixture, which is stirred for 5 minutes.

### EXAMPLE 3

### Tacrolimus and a CYP3A4 Inhibitor Compound

The following tablet compositions may be prepared:

### Composition 3A:

| Substance | | Mg | |
|---|---|---|---|
| Tacrolimus | Active ingredient | 2.0 | |
| Spiro ortho ester | CYP3A4 inhibitor | 2.0 | |
| Lactose 200 mesh | Carrier | 41.7 | |
| PEG 6000 | Vehicle | 34.3 | |
| Poloxamer 188 | Vehicle | 14.7 | |
| Hypromellose USP (Metolose | Filler | 61.8 | |
| 90SH) | | | |
| Dimeticone 350 | Antioxidant | 0.25 | microgram |
| Citric acid monohydrate | Antioxidant | 25 | microgram |
| Isopropyl alcohol | Antioxidant | 0.5 | microL |
| Butyl hydroxy toluene (BHT) | Antioxidant | 5 | microgram |
| Total | | 100.0 | |

### Composition 3B:

| Substance | | Mg |
|---|---|---|
| Tacrolimus | Active ingredient | 2.0 |
| Spiro ortho ester | CYP3A4 inhibitor | 2.0 |
| Lactose monohydrate 125 mesh | Carrier | 14.3 |
| Rylo MD50 (glyceryl monostearate) | Vehicle | 64.7 |
| Pharmacoat 606 | Carrier | 14.3 |
| Pharmatose DCL14 | Filler | 105.8 |
| Talc:Magnesium stearate | Glidant | 9.52:1.06 |
| Total | | 100.0 |

The CYP3A4 inhibitor of compositions 3A and 3B is be substituted with 5 or 10 mg of limonin or 200 mg of ketoconazole, or with 5, 10 or 200 mg of cyclosporine A.

### EXAMPLE 4

### Preparation of a Delayed Release Tacrolimus Formulation (Granulate)

The following granulate was prepared:

Composition (dosage form)

| Ingredient | mg |
|---|---|
| Tacrolimus | 2 |
| Lactose | 80 |
| PEG 6000 | 15 |
| Poloxamer 188 | 6 |
| Metolose SH 90 | 80 |
| Avicel PH200 | 60 |
| Total | 243 |

The formulation was based on melt granulation in a high shear mixer Pellmix 1/8. 16 g micronized tacrolimus was mixed with 640 g lactose 125 mesh and 120 g polyethylene glycol 6000, 48 g Poloxamer 188 and 640 g hydroxypropylmethylcellulose (hypromellose) Metolose SH 90 15.000 cP in the high shear mixer. The jacket of the mixer bowl was heated to 80° C. and the blend was heated at an impeller rotation speed of 1000 rpm until melting point of PEG and Poloxamer. After melting the kneading was continued for 4 minutes at 800 rpm. The granulated was sieved through sieve size of 0.7 mm and cooled on a tray. The granulate was mixed with 480 g Avicel PH200 for 3 minutes. The mixture may be compressed into tablets after addition of magnesium stearate and a granulate comprising a CYP3A4 inhibitor or more preferred cyclosporine A.

### EXAMPLE 5

### Clinical Study According to the Invention

It appears from the clinical study that administration of tacrolimus and cyclosporine in the ratio of 1:2 results in an increase of bioavailability of tacrolimus of 30% and when administered in a ratio of 1:20, the increase in tacrolimus bioavailability of tacrolimus is above 100%

| | |
|---|---|
| Study | Bioavailability increase of tacrolimus by co-administration |
| | with cyclosporine A |
| SPECIALITY NAME | Tacrolimus/Cyclosporine A |
| STUDY TITLE | A three - way crossover pilot bioavailability study of two |
| | different combinations of Prograf ® andNeoral ® vs. the |
| | marketed reference Prograf ® in male healthy volunteers. |
| METHODOLOGY | |
| Clinical | An open-label, laboratory-blind, single-dose, randomized |
| | three-period cross-over pilot study under fasted conditions |
| | with a minimum washout period of 10 days between |
| | treatments. Blood sampling points: predose, 0.5 h, 1 h, 1.5 h, |
| | 2 h, 3 h, 4 h, 6 h, 8 h, 10 h, 12 h, 16 h, 24 h, 48 h, 72 h and 96 |
| | hours post-dose (16 samples per pharmacokinetic profile). |
| | All administrations will be given in the morning. |
| Analytical | Limit of Quantification of ca. 0.5 ng/ml for tacrolimus and |
| | cyclosporine A. |
| Statistical | Statistics according to guideline CPMP/EWP/QWP/1401/98: |
| | The statistical method is based upon the 90% confidence |
| | interval for the ratio of the population means (fasted), |
| | equivalent to the two one-sided tests procedure, using |
| | ANOVA for the logarithmic transformed AUC and Cmax |
| | values, and using non-parametric methods for the |
| | untransformed tmax values. |
| SUBJECTS | 12 healthy male subjects, aged between 18 and 65 years |
| TREATMENT A | |
| Name | Prograf ® Tacrolimus 5 mg |
| Pharm. Formulation | Capsule |
| Administration route | Oral once under fasted condition |
| Dose administered | 1 × 5 mg = 5 mg Tacrolimus |
| Manufacturer | Prograf ®: Fujisawa AG (Switzerland) |
| TREATMENT B | |
| Name | Prograf ® Tacrolimus 5 mg/Sandimmune Neoral ® CsA |
| Pharm. Formulation | 10 mg |
| Administration route | Capsule |
| Dose administered | Oral once under fasted condition |
| Manufacturer | 1 × 5 mg = 5 mg Tacrolimus; 1 × 10 mg = 10 mg Cyclosporine A |
| | Prograf ®: Fujisawa AG (Switzerland) |
| | Neoral ®: Novartis AG (Switzerland) |
| REFERENCE | |
| TREATMENT C | |
| Name | Prograf ® Tacrolimus 5 mg/Neoral CsA 100 mg |
| Pharm. Formulation | Capsules |
| Administration route | Oral once under fasted condition |
| Dose administered | 1 × 5 mg = 5 mg Tacrolimus; 1 × 100 mg = 100 mg |
| Manufacturer | Cyclosporine A |
| | Prograf ®: Fujisawa AG (Switzerland) |
| | Neoral ®: Novartis AG (Switzerland) |
| Treatment duration | One single dose of Prograf ® Tacrolimus capsules 1 × 5 mg/ |
| | under fasted condition |
| | One single dose of Prograf ® Tacrolimus capsules 1 × 5 mg/ |
| | Neoral ® CsA 1 × 10 mg, under fasted conditions |
| | One single dose of Prograf ® Tacrolimus capsules |
| | 1 × 5 mg/Neoral ® CsA 1 × 100 mg under fasted conditions. |
| Evaluation criteria | Pharmacokinetic parameters: |
| | AUC (0-tn), AUC (0-inf), Cmax, % AUC, tmax, λz, t½ |
| | Bioequivalence will be evaluated by means of the |
| | bioequivalence statistics: |
| | AUC (0-tn) - ratio including the 90% interval for this |
| | measure of relative bioavailability |
| | AUC (0-inf) - ratio including the 90% interval for this |
| | measure of relative bioavailability |
| | Cmax - ratio including the 90% interval for this measure of |
| | rate of absorption |
| | tmax - difference of medians (i.e. Hodge-Lehmann point |
| | estimator) including the non-parametric 90% confidence |
| | interval for this measure of rate of absorption |
| Results | See tables below |

### Pharmacokinetic Data

### Tacrolimus:

| | | |
|---|---|---|
| Treatment A | Average of Half life | 33,98235908 |
| | StdDev of Half life | 8,294726752 |
| | Average of Cmax (ng/ml) | 29,63333333 |
| | StdDev of Cmax (ng/ml) | 6,806992839 |
| | Average of AUClast (hr * ng/ml) | 207,0758032 |
| | StdDev of AUClast (hr * ng/ml) | 77,41077978 |
| | Average of AUCINF_obs (hr * ng/ml) | 245,7029899 |
| | StdDev of AUCINF_obs (hr * ng/ml) | 84,97476415 |
| Treatment B | Average of Half life | 30,38285487 |
| | StdDev of Half life | 8,130724359 |
| | Average of Cmax (ng/ml) | 39,49166667 |
| | StdDev of Cmax (ng/ml) | 10,12893769 |
| | Average of AUClast (hr * ng/ml) | 274,5913061 |
| | StdDev of AUClast (hr * ng/ml) | 112,2261623 |
| | Average of AUCINF_obs (hr * ng/ml) | 311,9256366 |
| | StdDev of AUCINF_obs (hr * ng/ml) | 119,7524699 |
| Treatment C | Average of Half life | 34,71691424 |
| | StdDev of Half life | 8,426821378 |
| | Average of Cmax (ng/ml) | 56,89166667 |
| | StdDev of Cmax (ng/ml) | 11,19078016 |
| | Average of AUClast (hr * ng/ml) | 407,7287845 |
| | StdDev of AUClast (hr * ng/ml) | 101,469039 |
| | Average of AUCINF_obs (hr * ng/ml) | 464,5356701 |
| | StdDev of AUCINF_obs (hr * ng/ml) | 119,920474 |
| Treatment B | Average of Cmax (ng/ml) | 28,86666667 |
| | StdDev of Cmax (ng/ml) | 6,192419608 |
| | Average of AUClast (hr * ng/ml) | 72,5017442 |
| | StdDev of AUClast (hr * ng/ml) | 20,86156547 |
| | Average of AUCINF_obs | 76,54695219 |
| | (hr * ng/ml) | |
| | StdDev of AUCINF_obs | 21,9413598 |
| | (hr * ng/ml) | |
| Treatment C | Average of Cmax (ng/ml) | 819,9166667 |
| | StdDev of Cmax (ng/ml) | 83,43692047 |
| | Average of AUClast (hr * ng/ml) | 2349,537236 |
| | StdDev of AUClast (hr * ng/ml) | 322,3928168 |
| | Average of AUCINF_obs | 2414,557329 |
| | (hr * ng/ml) | |
| | StdDev of AUCINF_obs | 343,2371777 |
| | (hr * ng/ml) | |

Ratio of Tacrolimus with Low Dose Cyclosporine A vs Tacrolimus Alone

| | | CsA + | Tac | | | |
|---|---|---|---|---|---|---|
| Dependent | Units | Tac mg | Ref mg | Ratio[% Ref] | CI_90_Lower | CI_90_Upper |
| Ln(Cmax) | ng/ml | 10 + 5 | 5 | 133.25 | 114.15 | 155.54 |
| Ln(AUCINF_obs) | hr * ng/ml | 10 + 5 | 5 | 125.10 | 97.22 | 160.97 |
| Ln(AUCall) | hr * ng/ml | 10 + 5 | 5 | 130.00 | 99.13 | 170.48 |

Ratio of Tacrolimus with High Dose Cyclosporine A vs Tacrolimus Alone

| | | CsA + | Tac | | | |
|---|---|---|---|---|---|---|
| Dependent | Units | Tac mg | Ref mg | Ratio[% Ref] | CI_90_Lower | CI_90_Upper |
| Ln(Cmax) | ng/ml | 100+ | 5 | 193.85 | 166.07 | 226.28 |
| Ln(AUCINF_obs) | hr * ng/ml | 100 + 5 | 5 | 194.26 | 150.96 | 249.96 |
| Ln(AUCall) | hr * ng/ml | 100 + 5 | 5 | 204.61 | 156.02 | 268.32 |

1. A pharmaceutical composition comprising tacrolimus or an analogue thereof and at least one second substance selected from the group of substances being a substrate for CYP3A4 and/or P-glycoprotein.

2. A pharmaceutical composition according to claim 1, wherein the at least one second substance is selected from the group of substances being a substrate and an inhibitor of CYP3A4 and/or P-glycoprotein.

3. A pharmaceutical composition according to claim 1, wherein the at least one second substance is selected from the group of substances being a substrate for and inhibitor of both CYP3A4 and P-glycoprotein.

4. A pharmaceutical composition according to claim 1, comprising a ratio of tacrolimus to the one second substance in a range of 1:0.1 to 1 to 200, preferable a ratio in the range of 1:0.5 to 1:100, such as in the range of 1:1 to 1:50, such as a ratio in range of 1:1.5 to 1:25 such as a ratio in the range of 1:2 to 1:20.

5. A pharmaceutical composition according to claim 1, comprising cyclosporine A as the at least one second substance.

6. A pharmaceutical composition according to claim 1, which upon administration to a human results in an increased bioavailability of tacrolimus or an analogue thereof measured as the area under the concentration/time curve (AUCᵢₙᵥₑₙₜᵢₒₙ) compared with the bioavailability of the tacrolimus or the analogue thereof obtained under similar conditions with administration of a similar pharmaceutical composition not comprising said at lest one second substance (AUCₛₑₚₐᵣₐₜₑ).

7. A pharmaceutical composition according to claim 6 wherein the increased bioavailability of tacrolimus results in a relative AUCᵢₙᵥₑₙₜᵢₒₙ/AUCₛₑₚₐᵣₐₜₑ ratio value of at least about 1.1.

8. The pharmaceutical composition according to claim 7, wherein the relative AUCᵢₙᵥₑₙₜᵢₒₙ/AUCₛₑₚₐᵣₐₜₑ value is at least about 1.2, or at least about 1.3, or at least about 1.4, or at least about 1.5 or at least about 1.6.

9. A pharmaceutical composition according to claim 1, wherein the stability of tacrolimus or the analogue thereof after a storage period of at least 3 months of storage at a temperature of about 25° C. and a relative humidity of about 60% and/or at a temperature of about 40° C. and a relative humidity of about 75% results in the recovery of at least 90%, or at least 95%, or at least 98% such as at least 99% or at least about 100%, relative to the assayed amount prior to storage.

10. A pharmaceutical composition according to claim 9 wherein the stability period is at least 6 months or at least 9 months or at least 12 month or at least 18 months or at least 24 months.

11. A pharmaceutical composition according to claim 1, wherein physical form of tacrolimus or the analogue thereof is crystalline, amorphous or combinations thereof.

12. A pharmaceutical composition according to claim 1, wherein the tacrolimus or analogue thereof is present in the composition as a solid dispersion or a solid solution.

13. A pharmaceutical composition according to claim 11, wherein at least 10%, such as at least 20%, such as at least 30%, such as at least 40%, such as at least 50%, such as at least 60%, such as at least 70%, such as at least 80%, such as at least 90%, such as at least 95% is in amorphous form.

14. A pharmaceutical composition according to claim 13, wherein the tacrolimus is substantially in the amorphous form.

15. A pharmaceutical formulation according to claim 1, wherein the vehicle for the tacrolimus or analogue thereof is a hydrophilic and/or water-miscible vehicle.

16. (canceled)

17. A pharmaceutical composition according to claim 1, further comprising a CYP3A4 inhibitor selected from the group consisting of diethyl dithiocarbamate, ketoconazole, itraconazole, erythromycin, ritonavir and lanzoprazol.

18. A pharmaceutical composition according to claim 1, further comprising a CYP3A4 inhibitor selected from the group consisting of safrole, rutaecarpine, limonin, dipiperamide A (from white pepper), gomisin C (from schisandra fruit), paradisin A and paradisin B (from grape fruit juice).

19.-26. (canceled)

27. A pharmaceutical composition according to claim 1, further comprising one or more pharmaceutically acceptable excipients.

28. The composition according to claim 27, wherein the pharmaceutically acceptable excipient(s) is/are selected from the group consisting of fillers, disintegrants, binders and lubricants.

29. The composition according to claim 1, wherein the tacrolimus is solubilized in propylene glycol ester or deglycerol monolaureate or a combination thereof.

30. The composition according to claim 1, comprising cyclosporine A and wherein the cyclosporine A is solubilized in propylene glycol ester or deglycerol monolaureate or a combination thereof.

31. A method for the preparation of a pharmaceutical composition according to claim 1, the method comprising the steps of dissolving or dispersing tacrolimus in a solid, hydrophilic or water-miscible vehicle to obtain a solid dispersion or a solid solution or a mixture thereof, followed by mixing the at least one second substance with the solid dispersion or solid solution.

32. A solid dosage form comprising the pharmaceutical composition according to claim 1.

33. The dosage form according to claim 32, which is a unit dosage form.

34. The dosage form according to claim 33, which further comprises a pharmaceutically acceptable additive selected from the group consisting of flavoring agents, coloring agents, taste-masking agents, pH-adjusting agents, buffering agents, preservatives, stabilizing agents, anti-oxidants, wetting agents, humidity-adjusting agents, surface-active agents, suspending agents and absorption enhancing agents.

35. The dosage form according to claim 34 for the preparation of a medicament in the form of tablets, capsules, sachets, granules, pellets, micro-spheres or nanoparticles.

36. A single solid dosage form for oral administration comprising a first solid pharmaceutical composition containing tacrolimus or an analogue thereof as the active substance and a second solid pharmaceutical composition containing at least one second substance selected from the group of substances being a substrate for CYP3A4 and/or P-glycoprotein, wherein the first and the second pharmaceutical composition are present in separate entities.

37. The dosage form according to claim 36, wherein the second substance is cyclosporine A.

38. The dosage form according to claim 37, further comprising a CYP3A4 inhibitor being selected from the group consisting of diethyl dithiocarbamate, ketoconazole, itraconazole, erythromycin, ritonavir, lanzoprazol, safrole, rutaecarpine, limonin, dipiperamide A (from white pepper), gomisin C (from schisandra fruit), paradisin A and paradisin B (from grape fruit juice).

39.-44. (canceled)

45. The dosage form according to claim 36, wherein the first solid pharmaceutical composition is in the form of granulate, granules, grains, beads or pellets.

46. The dosage form according to claim 36, wherein the second solid pharmaceutical composition is in the form of granulate, granules, grains, beads or pellets.

47. The dosage form according to claim 45 or 46, wherein the granulate, granules, grains, beads or pellets are entero-coated.

48. The dosage form according to claim 36, wherein the dosage form is a tablet.

49. The dosage form according to claim 48, in which the first and second pharmaceutical compositions are present in at least two separate layers optionally separated by an intermediate, inactive layer.

50. The dosage form according to claim 36, which is a tablet prepared by compressing the first pharmaceutical composition in the form of granulate together with the second pharmaceutical composition in the form of granulate having a protective coating.

51. The dosage form according to claim 36, which is a tablet prepared by compressing the first pharmaceutical composition in the form of granulate together with the second pharmaceutical composition in the form of entero-coated granulate.

52. The solid dosage form according to claim 36, wherein the tacrolimus or the analogue thereof and the second substance after a storage period of at least 3 months of storage at a temperature of about 25° C. and a relative humidity of about 60% and/or at a temperature of about 40° C. and a relative humidity of about 75% results in the recovery of at least 90%, or at least 95%, or at least 98% such as at least 99% or at least about 100%, relative to the assayed amount prior to storage.

53. A single solid dosage form suitable for oral administration comprising tacrolimus or an analogue thereof as the active substance and a second solid pharmaceutical composition containing a substrate for CYP3A4 and/or P-glycoprotein as the second active substance, wherein the tacrolimus is present as an active ingredient either of an immediate release pharmaceutical formulation, an entero-coated immediate release pharmaceutical formulation or of a delayed release pharmaceutical formulation; and the second active substance is present as an active ingredient of an immediate release pharmaceutical formulation, an entero-coated immediate release pharmaceutical formulation or of a delayed release pharmaceutical formulation.

54. A single solid dosage form according to claims 53, wherein the second active substance is cyclosporine A.

55. A method for preparing a single solid dosage form comprising a first solid pharmaceutical composition containing tacrolimus as the active substance and second solid pharmaceutical composition containing an substrate for CYP3A4 and/or P-glycoprotein as the active substance, the first and the second pharmaceutical composition being present in separate entities, which method comprising the steps of:
i) preparing the first solid pharmaceutical composition,
ii) preparing the second solid pharmaceutical composition, and
iii) compressing the first and second compositions into a multilayer tablet, the first and second compositions being present in separate layers.

56. A method for treating a patient in need thereof by coadministration of tacrolimus or an analogue thereof with at least one substance selected from the group of substances being a substrate for CYP3A4 and/or P-glycoprotein.

57. A method according to claim 56, wherein the substance being a substrate for CYP3A4 and/or P-glycoprotein is also an inhibitor of CYP3A4 and/or P-glycoprotein.

58. A method according to claim 56, wherein the substance being a substrate for CYP3A4 and/or P-glycoprotein is a substrate of both CYP3A4 and P-glycoprotein.

59. A method according to claim 56, wherein the substance being a substrate for CYP3A4 and/or P-glycoprotein is cyclosporine A.

60. A method for increasing the bioavailability of tacrolimus, comprising co-administering a dosage of tacrolimus and a dosage of cyclosporine to a patient in need of immunosuppression by tacrolimus, wherein the ratio of tacrolimus to cyclosporine administrated is in a range of 1:0.1 to 1 to 200, preferable a ratio in the range of 1:0.5 to 1:100, such as in the range of 1:1 to 1:50, such as a ratio in range of 1:1.5 to 1:25 such as a ratio in the range of 1:2 to 1:20.
